(19) United States
(12) Patent Application Publication
(10) Pub. No.: US 2025/0283085 A1
(43) Pub. Date: Sep. 11, 2025

(54) Oligonucleotide Terminal Phosphate Derivatives and Method of Use

(71) Applicant: SynerK Biotech Limited, Hong Kong (HK)

(72) Inventors: Dong YU, Westboro, MA (US); Weiwen JIANG, Winchester, MA (US); Tao LAN, Concord, MA (US)

(21) Appl. No.: 19/073,552

(22) Filed: Mar. 7, 2025

Related U.S. Application Data
(60) Provisional application No. 63/563,908, filed on Mar. 11, 2024.

(51) Int. Cl. C12N 15/113; C07H 19/067

(52) U.S. Cl. CPC C12N 15/113; C07H 19/067; C12N 2310/11; C12N 2310/14; C12N 2310/31; C12N 2310/321; C12N 2310/322; C12N 2310/351

(57) ABSTRACT

The present invention provides a nucleotide derivative that stabilizes the terminal monophosphate of an oligonucleotide and oligonucleotides comprising the derivative. This monophosphate derivative according to the invention can be used to improve the targeted gene silencing efficiency of oligonucleotide drugs and has broad application prospects for drug research and development.

### RELATED APPLICATION

This application claims the benefit of U.S. Provisional Application No. 63/563,908, filed on Mar. 11, 2024. The entire teachings of the above application are incorporated herein by reference.

### BACKGROUND

The 5′-monophosphate at the end of the siRNA guide strand is critical for RISC recognition. The phosphorylation of the 5′-hydroxyl group plays a certain role in whether siRNA can be effectively loaded onto Argonaute-2 (Ago2) inside the cell. The 5′-terminal monophosphate of the guide strand in siRNA has an H-bond interaction with Ago2, thereby ensuring accurate positioning and precise cleavage of the target mRNA. Since the 5′-terminal monophosphate group could be easily hydrolyzed by phosphatases in vivo, the siRNA's cleavage activity on the target mRNA is dependent on the stability of the 5′-terminal monophosphate of guide strand.

Thus, there is a need for monophosphate derivatives that are stable in phosphatase media.

### SUMMARY OF THE INVENTION

The present disclosure aims to solve the existing problems of the prior art and provide monophosphate derivatives, oligonucleotides comprising the monophosphate derivative of the invention, a conjugate thereof and uses thereof.

The present invention solves the above problems by providing a monophosphate derivative as described herein as the 3′ terminal nucleotide or the 5′-terminal nucleotide to the antisense strand or the sense (guide chain) strand of siRNA. Preferably, the monophosphate derivative as described herein is the 3′ terminal nucleotide of the sense strand. Preferably, the monophosphate derivative as described herein is the 3′ terminal nucleotide of the antisense strand. Preferably, the monophosphate derivative as described herein is the 5′ terminal nucleotide of the sense strand. Preferably, the monophosphate derivative as described herein is the 5′ terminal nucleotide of the antisense strand.

The present invention solves the above problems by providing a monophosphate derivative as described herein as 3′ terminal nucleotide or the 5′-terminal nucleotide of an antisense oligonucleotide. Preferably, the monophosphate derivative as described herein is the 3′ terminal nucleotide of the antisense oligonucleotide. Preferably, the monophosphate derivative as described herein is the 5′ terminal nucleotide of the antisense oligonucleotide.

The present invention also relates to the synthesis and use of a type of phosphate ester derivatives containing sulfur or nitrogen at the 3′ or 5′-terminal end, preferably the 5′-terminal end. It is very important to synthesize monophosphate derivatives that are stable in phosphatase media.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows knockdown of SOD1 with siRNAs.

FIG. 2 shows knockdown of SOD1 with siRNAs.

FIG. 3 shows knockdown of SOD1 with siRNAs.

FIG. 4 shows knockdown of SOD1 with siRNAs.

FIG. 5 shows knockdown of MAPT with siRNAs.

### DETAILED DESCRIPTION

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point value of the various ranges, and the individual point values may be combined with one another to produce one or more new numerical ranges, which should be deemed to have been specifically disclosed herein.

In the present disclosure, the number of carbon atom(s) for C₁-C₁₀ group may be 1, 2, 3, 4, 5, 6, 7, 8, 9, or 10; the number of carbon atoms for C₆-C₁₀ group may be 6, 7, 8, 9, or 10; the number of carbon atoms for C₅-C₁₀ group may be 5, 6, 7, 8, 9, or 10.

In the present disclosure, the integers of 1-10 include 1, 2, 3, 4, 5, 6, 7, 8, 9 or 10; the integers of 1-4 include 1, 2, 3 or 4; the integers of 1-6 include 1, 2, 3, 4, 5 or 6.

Unless a point of attachment indicates otherwise, the chemical moieties listed in the definitions of the variables of Formula (I) of this disclosure, and all the embodiments thereof, are to be read from left to right, wherein the right hand side is directly attached to the parent structure as defined. However, if a point of attachment (e.g., a dash “-”) is shown on the left hand side of the chemical moiety (e.g., —C₁-C₆alkyl-N(R⁶)₂), then the left hand side of this chemical moiety is attached directly to the parent moiety as defined.

“Alkyl,” by itself, or as part of another substituent, means, unless otherwise stated, a straight or branched chain hydrocarbon, having the number of carbon atoms designated (i.e., C₁-C₆ means one to six carbons). Representative alkyl groups include straight and branched chain alkyl groups having 1, 2, 3, 4, 5, 6, 7, 8, 9, or 10 carbon atoms. Further representative alkyl groups include straight and branched chain alkyl groups having 1, 2, 3, 4, 5, 6, 7 or 8 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like. For each of the definitions herein (e.g., alkyl, alkoxy, arylalkyl, cycloalkylalkyl, heterocycloalkylalkyl, heteroarylalkyl, etc.), when a prefix is not included to indicate the number of carbon atoms in an alkyl portion, the alkyl moiety or portion thereof will have 10 or fewer main chain carbon atoms or 8 or fewer main chain carbon atoms or 6 or fewer main chain carbon atoms. For example, C₁-C₆alkyl refers to a straight or branched hydrocarbon having 1, 2, 3, 4, 5 or 6 carbon atoms and includes, but is not limited to, —CH₃, C₂alkyl, C₃alkyl, C₄alkyl, C₅alkyl, C₆alkyl, C₁-C₂alkyl, C₂alkyl, C₃alkyl, C₁-C₃alkyl, C₁.

C₄alkyl, C₁-C₅alkyl, C₁-C₆alkyl, C₂-C₃alkyl, C₂-C₄alkyl, C₂-C₅alkyl, C₂-C₆alkyl, C₃-C₄alkyl, C₃. C₅alkyl, C₃-C₆alkyl, C₄-C₅alkyl, C₄-C₆alkyl, C₅-C₆ alkyl and C₆alkyl. While it is understood that substitutions are attached at any available atom to produce a stable compound, when optionally substituted alkyl is an R group of a moiety such as —OR (e.g. alkoxy), —SR (e.g. thioalkyl), —NHR (e.g. alkylamino), —C(O)NHR, and the like, substitution of the alkyl R group is such that substitution of the alkyl carbon bound to any O, S, or N of the moiety (except where N is a heteroaryl ring atom) excludes substituents that would result in any O, S, or N of the substituent (except where N is a heteroaryl ring atom) being bound to the alkyl carbon bound to any O, S, or N of the moiety.

“Alkoxy” or “alkoxyl” refers to a —O-alkyl group, where alkyl is as defined herein. By way of example, “C₁-C₆alkoxy” refers to a —O—C₁-C₆alkyl group, where alkyl is as defined herein. While it is understood that substitutions on alkoxy are attached at any available atom to produce a stable compound, substitution of alkoxy is such that O, S, or N (except where N is a heteroaryl ring atom), are not bound to the alkyl carbon bound to the alkoxy O. Further, where alkoxy is described as a substituent of another moiety, the alkoxy oxygen is not bound to a carbon atom that is bound to an O, S, or N of the other moiety (except where N is a heteroaryl ring atom), or to an alkene or alkyne carbon of the other moiety.

“Alkylene” by itself or as part of another substituent means a linear or branched saturated divalent hydrocarbon moiety derived from an alkane having the number of carbon atoms indicated in the prefix. For example, (i.e., C₁-C₆ means one to six carbons; C₁-C₆alkylene is meant to include methylene, ethylene, propylene, 2-methylpropylene, pentylene, hexylene and the like). C₁₋₄ alkylene includes methylene —CH₂—, ethylene —CH₂CH₂—, propylene —CH₂CH₂CH₂—, and isopropylene —CH(CH₃)CH₂—, —CH₂CH(CH₃)—, —CH₂—(CH₂)₂CH₂—, —CH₂—CH(CH₃)CH₂—, —CH₂—C(CH₃)₂—CH₂—CH₂CH(CH₃)—. Typically, an alkyl (or alkylene) group will have from 1 to 24 carbon atoms, with those groups having 10 or fewer, 8 or fewer, or 6 or fewer carbon atoms. When a prefix is not included to indicate the number of carbon atoms in an alkylene portion, the alkylene moiety or portion thereof will have 12 or fewer main chain carbon atoms or 8 or fewer main chain carbon atoms, 6 or fewer main chain carbon atoms, or 4 or fewer main chain carbon atoms, or 3 or fewer main chain carbon atoms, or 2 or fewer main chain carbon atoms, or 1 carbon atom.

“Alkenyl” refers to a linear monovalent hydrocarbon radical or a branched monovalent hydrocarbon radical having the number of carbon atoms indicated in the prefix and containing at least one double bond. For example, C₂-C₆ alkenyl is meant to include ethenyl, propenyl, and the like.

“Halogen” or “halo” refers to all halogens, that is, chloro (Cl), fluoro (F), bromo (Br), or iodo (I). The term “haloalkyl” refers to an alkyl substituted by one to seven halogen atoms. Haloalkyl includes monohaloalkyl or polyhaloalkyl. For example, the term “C₁-C₆haloalkyl” is meant to include trifluoromethyl, difluoromethyl, 2,2,2-trifluoroethyl, 4-chlorobutyl, 3-bromopropyl, and the like.

The term “haloalkoxy” refers to an alkoxy substituted by one to seven halogen atoms. Haloalkoxy includes monohaloalkoxy or polyhaloalkoxy. For example, the term “C₁-C₆haloalkoxy” is meant to include trifluoromethoxy, difluoromethoxy, 2,2,2-trifluoroethoxy, 4-chlorobutoxy, 3-bromopropoxy, and the like.

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5-position. A 5-methyl cytosine is a modified nucleobase.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases refer to nucleobases that are capable of forming hydrogen bonds with one another.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, the phrase “inhibiting the expression or activity” refers to a reduction or blockade of the expression or activity relative to the expression of activity in an untreated or control sample and does not necessarily indicate a total elimination of expression or activity.

As used herein, “mRNA” means an RNA transcript that encodes a protein and includes pre-mRNA and mature mRNA unless otherwise specified.

As used herein “internucleoside linkage” means a covalent linkage between adjacent nucleosides in an oligonucleotide. As used herein “naturally occurring internucleoside linkage” means a 3′ to 5′ phosphodiester linkage. As used herein, “modified internucleoside linkage” means any internucleoside linkage other than a naturally occurring internucleoside linkage. The nucleoside residues of the nucleic acid compound of the invention can be coupled to each other by any of the numerous known internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Methods of preparation of phosphorous-containing and non-phosphorous-containing internucleoside linkages are well known to those skilled in the art. Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages.

In some embodiments, the nucleic acid compound of the invention may comprise combinations of internucleotide linkages. In some embodiments, the nucleic acid compound of the invention may comprise combinations of phosphorothioate and phosphodiester internucleotide linkages. In some embodiments more than half but less that all of the internucleotide linkages are phosphorothioate internucleotide linkages. In some embodiments all of the internucleotide linkages are phosphorothioate internucleotide linkages.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “MOE” means methoxyethyl. “2′-MOE” means a 2′-OCH₂CH₂OCH₃ group in place of the 2′ OH group of a ribosyl sugar moiety.

As used herein, “OMe” means methoxy. “2′-OMe” means a 2′-OCH₃ group in place of the 2′ OH group of a ribosyl sugar moiety.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “self-complementary” in reference to an oligonucleotide means an oligonucleotide that at least partially hybridizes to itself.

As used herein, “sugar moiety” means an unmodified sugar moiety or a modified sugar moiety.

As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal. For example, a therapeutically effective amount improves a symptom of a disease.

As used herein, “treat”, “treatment”, or “treating” refers to administering a compound described herein to effect an alteration or improvement of a disease, disorder, or condition.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

In a first aspect, the present disclosure provides a monophosphate derivative comprising a structure represented by formula (A) or (B):

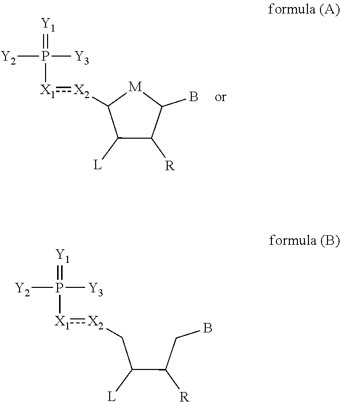

wherein:
X₁ is —CH₂—, —CHR^{a}—, —CR^{a}R^{b}—, —NH—, —NR^{c}—, —N═CH—, —N═CR^{d}—, or —S—; X₂ is —CH₂—, —CHR^{a}′—, —CR^{a}′R^{b}′—, —NH—, —NR^{c}′—, —CH═N—, —CR^{d}′=N—, or —S—;
R^{a}, R^{b}, R^{c}, R^{d}, R^{a}′, R^{b}′, R^{c}′, and R^{d}′ are independently selected from C₁-C₁₀ alkyl;
Y₁ is O or S;
Y₂ and Y₃ are independently selected from —OH, —O(CH₂)ᵥOC(O)R^{g}; —SR^{e}, —NHR^{e}, C₁-C₁₀ alkyl, or C₁-C₁₀ alkoxy, —ONa⁺, —SNa⁺, wherein v is 1-6;
B is selected from H, C, O, N or a nucleotide base;
M is selected from O, S, —CH₂, or —NH;
R is selected from H, —OH, —NH₂, halogen, C₁-C₁₀ alkyl, C₁-C₁₀ alkoxy, C₂-C₁₀ alkenyl, —CN, or haloalkyl, —CO₂H, —CO₂R^{e}, or —CONHR^{f};
L is a linking group; and
R^{e}, R^{f}, and R^{g} are independently selected from H, C₁-C₁₀ alkyl, C₁-C₁₀ alkoxy, or C₂-C₁₀ alkenyl.

Preferably, the C₁-C₁₀ alkyl, at each instance, is independently a C₁-C₆ alkyl. Preferably, the C₁-C₁₀ alkyl, at each instance, is independently a C₁-C₃ alkyl. Preferably, the C₁-C₁₀ alkyl, at each instance, is independently methyl or ethyl. Preferably, the C₁-C₁₀ alkyl, at each instance, is independently methyl. Preferably, the C₁-C₁₀ alkyl, at each instance, is independently ethyl. Preferably, the C₁-C₁₀ alkoxy, at each instance, is independently a C₁-C₆ alkoxy. Preferably, the C₁-C₁₀ alkyl, at each instance, is independently a C₁-C₃ alkoxy. Preferably, the C₁-C₁₀ alkoxy, at each instance, is independently methoxy, ethoxy or methoxyethoxy. Preferably, the C₁-C₁₀ alkoxy, at each instance, is independently methoxy. Preferably, the C₁-C₁₀ alkoxy, at each instance, is independently ethoxy. Preferably, the C₁-C₁₀ alkoxy, at each instance, is independently methoxyethoxy.

Preferably, the C₂-C₁₀ alkenyl, at each instance, is independently a C₂-C₆ alkenyl. Preferably, the C₂-C₁₀ alkenyl, at each instance, is independently a C₂-C₄ alkenyl. Preferably, the C₂-C₁₀ alkenyl, at each instance, is independently ethynyl, or propynyl. Preferably, the C₂-C₁₀ alkenyl, at each instance, is independently ethynyl. Preferably, the C₂-C₁₀ alkenyl, at each instance, is independently propynyl.

Preferably, the halogen, at each instance, is independently selected from fluorine (F), chlorine (Cl), bromine (Br), or iodine (I).

Preferably, B is H.

Preferably, B is an unmodified nucleotide base. Preferably, the unmodified nucleotide base is selected from adenine, uracil, thymine, guanine or cytosine.

Preferably, B is a modified nucleotide base. Modified nucleotides comprise at least one modification relative to unmodified RNA or DNA. That is, modified nucleotides comprise a modified sugar moiety and/or a modified nucleobase and/or at least one modified internucleoside linkage. Preferably, the modified nucleotide base is selected from 5-methyl-cytidine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

Preferably, M is O. Preferably, M is S. Preferably, M is —CH2. Preferably, M is —NH.

Preferably, R is H.

Preferably, R is selected from —OH, —NH₂, halogen, C₁-C₁₀ alkyl, C₁-C₁₀ alkoxy, C₁-C₁₀ alkenyl, —CN, haloalkyl, —CO₂H, —CO₂R^{e}, —CONHR^{f}.

Preferably, L is a linking group. The linking group links the monophosphate derivative of the invention to a phosphoroamidite or to another nucleotide, preferably within an oligonucleotide.

Preferably, L is selected from —OH, —OR₂, wherein R₂ is a phosphate or an analog thereof. In embodiments, R₂ has the formula —P(Q)(W)Z—, wherein Q is O or S, W is hydroxy, C₁-C₆ alkyl, C₁-C₆ alkoxy, SH, NRₕRᵢ, and Z is CHRᵢ, O, or S, wherein each Rh and Rᵢ are independently H, C₁-C₆ alkyl.

Preferably L is selected from —OP(═O)(OH)O—, —OP(═O)(SH)O—, —OP(═S)(OH)O—, —OP(═O)(CH₃)O—, —OP(═O)(CH₂CH₃)O—, —OP(═O)(OCH₃)O—, —OP(═O)(OCH₂CH₃)O—, —OP(═O)(NH₂)O—, —OP(═O)(NHCH₃)O—, —OP(═O)(NHCH₂CH₃)O—, —OP(═S)(CH₃)O—, —OP(═S)(CH₂CH₃)O—, —OP(═S)(OCH₃)O—, —OP(═S)(OCH₂CH₃)O—, —OP(═S)(NH₂)O—, —OP(═S)(NHCH₃)O—, —OP(═S)(NHCH₂CH₃)O—, or —OP(═S)(SH)O—.

Preferably, Y₁ is O. Preferably, Y₁ is S.

Preferably, Y₂ is selected from —OH, —SH, —NH₂, —ONa⁺, or —SNa⁺.

Preferably, Y₂ is selected from —CH₃, —CH₂CH₃, —OCH₃, —OCH₂CH₃, —SCH₃, —SCH₂CH₃, —NHCH₃, or —NHCH₂CH₃.

Preferably, Y₃ is selected from —OH, —SH, —NH2, —ONa⁺, or —SNa⁺.

Preferably Y³ is selected from —CH₃, —CH₂CH₃, —OCH₃, —OCH₂CH₃, —SCH₃, —SCH₂CH₃, —NHCH₃, or —NHCH₂CH₃.

Y₂ and Y₃ can be the same or different. Preferably, Y₂ and Y₃ are the same. Preferably, Y₂ and Y₃ are different.

Preferably, the monophosphate derivative has the structure represented by formula (A).

Preferably, the monophosphate derivative has the structure represented by formula (B).

Preferably the monophosphate derivative has a structure represented by formula (A1), (A2), (A3) or (A4):

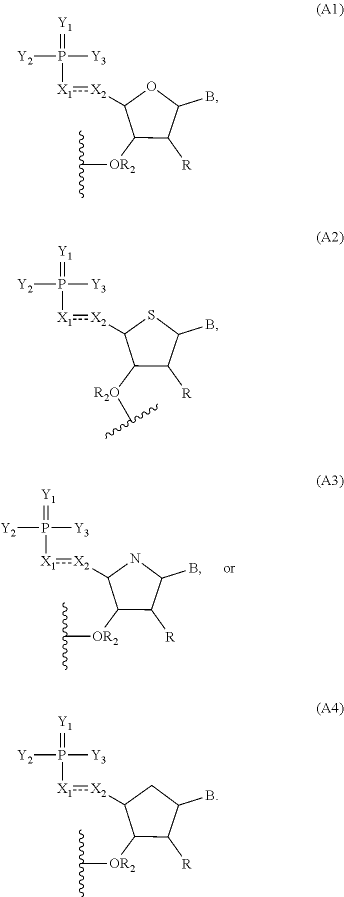

Preferably the monophosphate derivative has a structure represented by formula (A1).

Preferably the monophosphate derivative has a structure represented by formula (A2).

Preferably the monophosphate derivative has a structure represented by formula (A3).

Preferably the monophosphate derivative has a structure represented by formula (A4).

Preferably the monophosphate derivative has a structure represented by formula (B1):

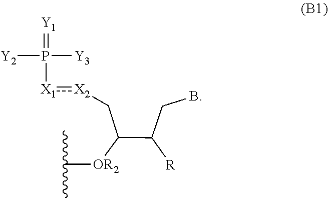

Preferably the monophosphate derivative has a structure represented by formula N1, N2, N3, N4, or N5. Preferably the monophosphate derivative has a structure represented by formula N2, N3, or N4:

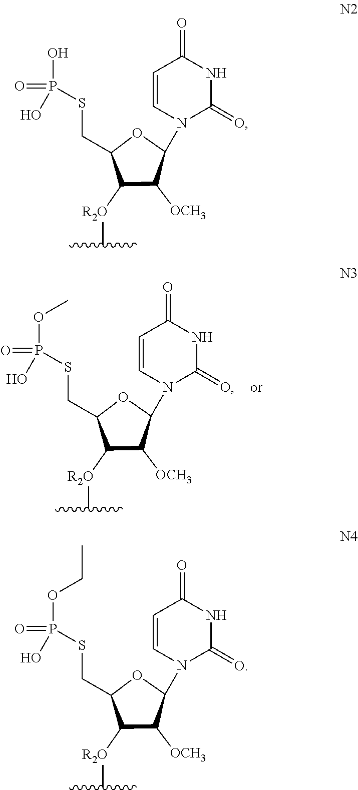

Preferably, the monophosphate derivative has a structure in Table 1.

TABLE 1

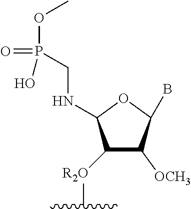

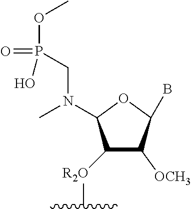

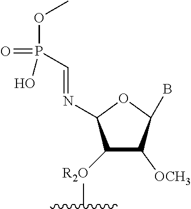

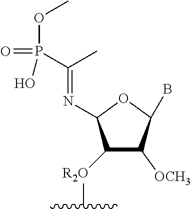

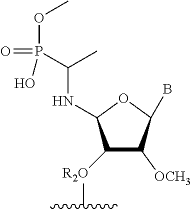

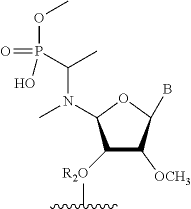

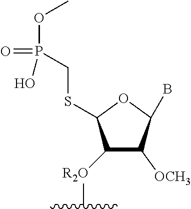

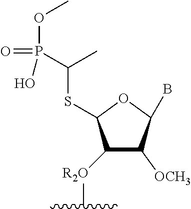

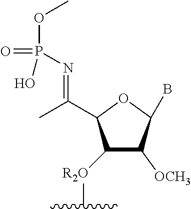

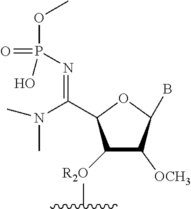

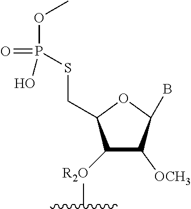

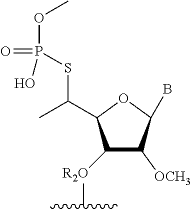

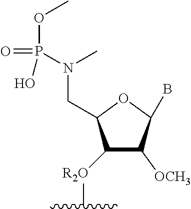

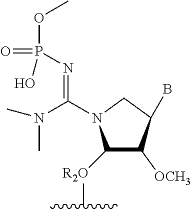

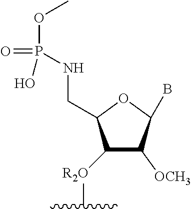

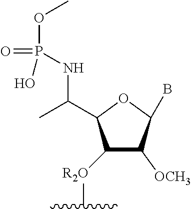

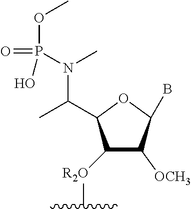

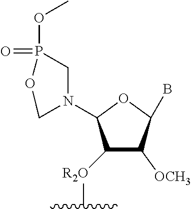

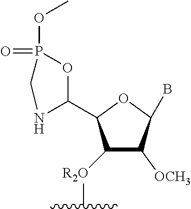

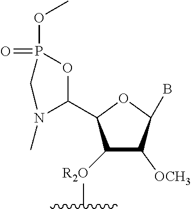

Preferably, the monophosphate derivative has a structure in Table 2.

TABLE 2

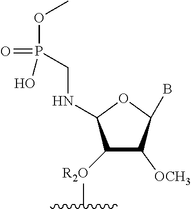

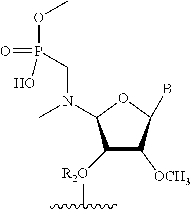

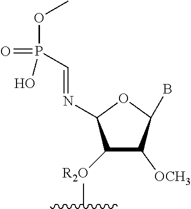

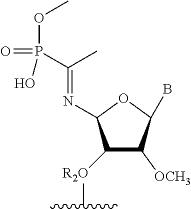

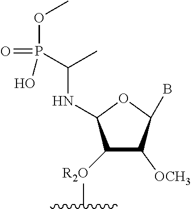

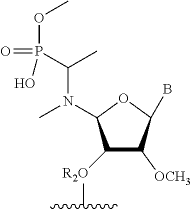

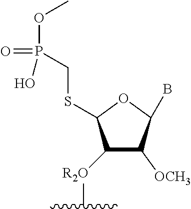

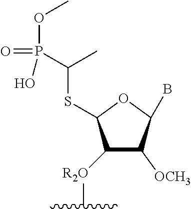

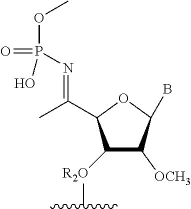

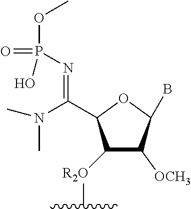

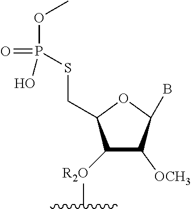

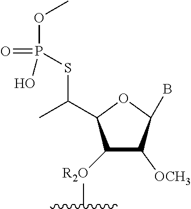

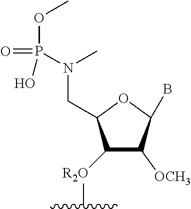

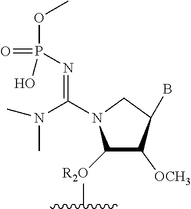

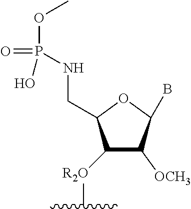

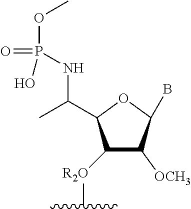

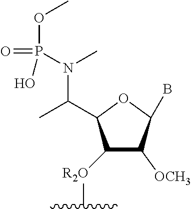

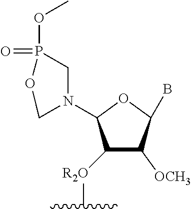

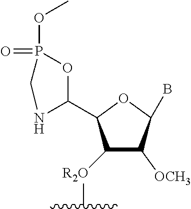

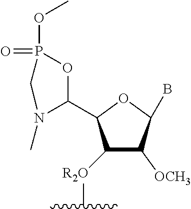

Preferably, the monophosphate derivative has a structure in Table 3.

TABLE 3

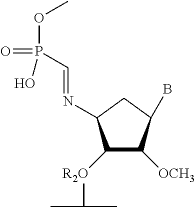

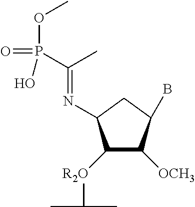

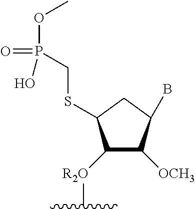

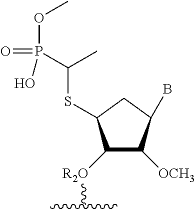

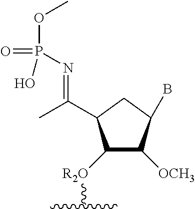

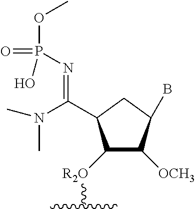

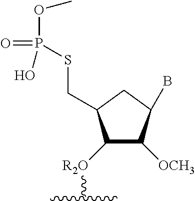

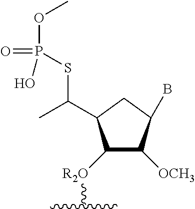

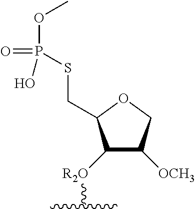

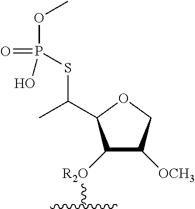

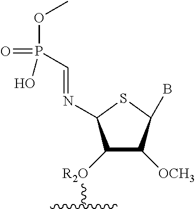

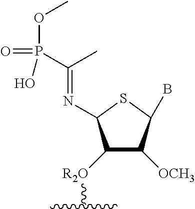

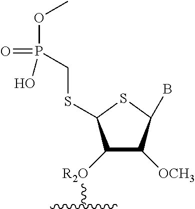

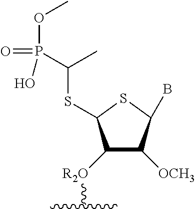

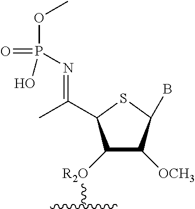

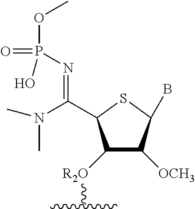

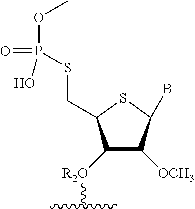

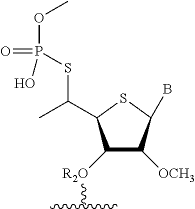

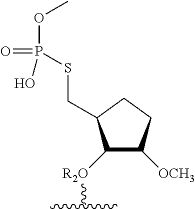

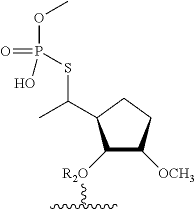

Preferably, the monophosphate derivative has a structure in Table 4.

TABLE 4

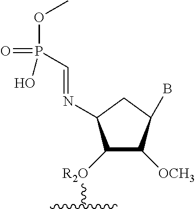

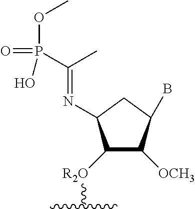

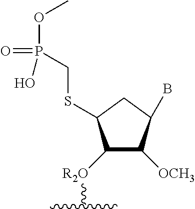

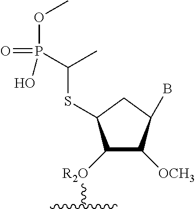

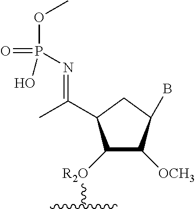

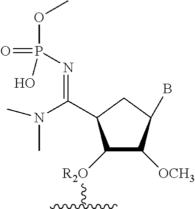

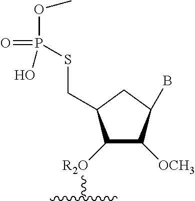

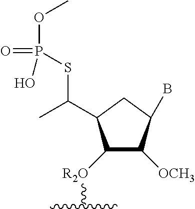

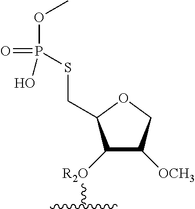

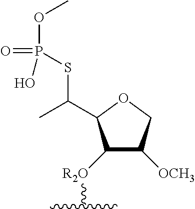

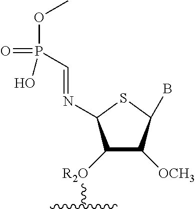

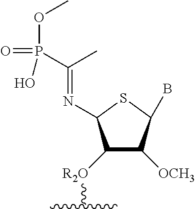

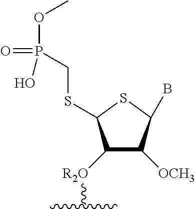

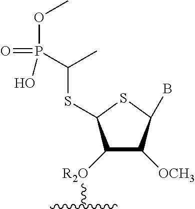

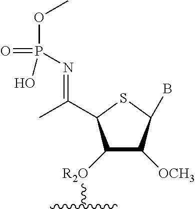

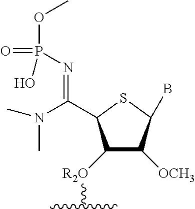

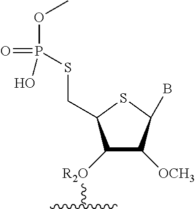

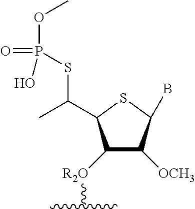

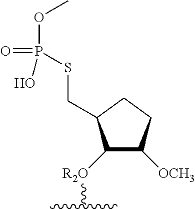

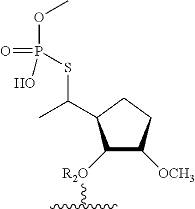

Preferably, the monophosphate derivative has a structure in Table 5.

TABLE 5

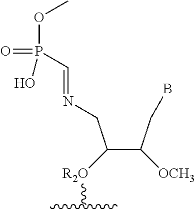

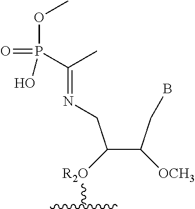

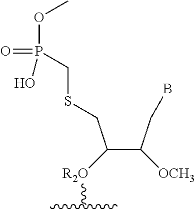

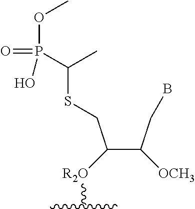

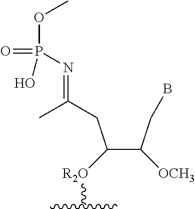

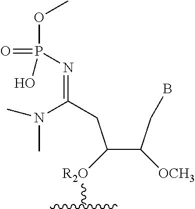

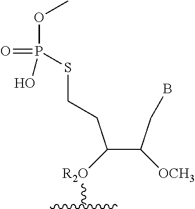

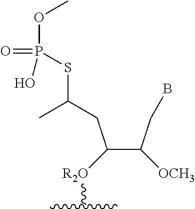

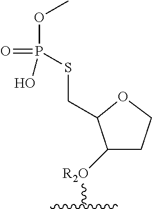

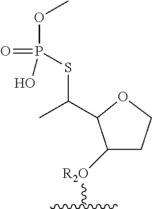

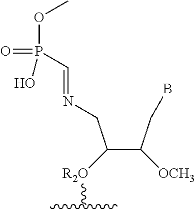

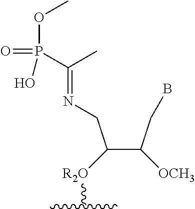

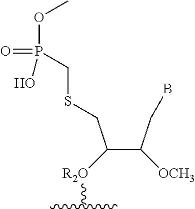

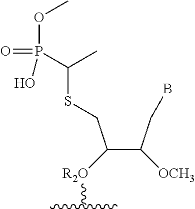

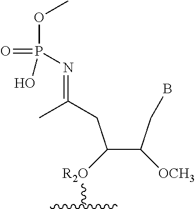

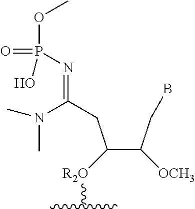

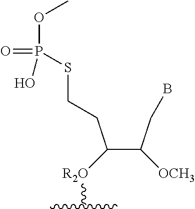

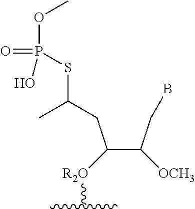

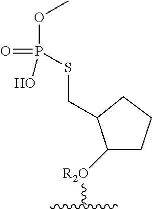

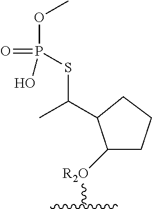

Preferably, the monophosphate derivative has a structure in Table 6.

TABLE 6

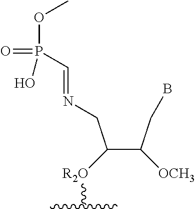

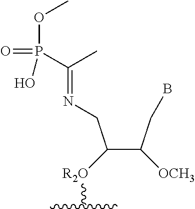

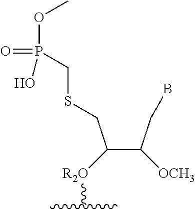

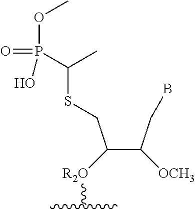

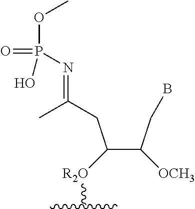

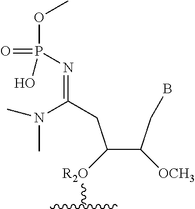

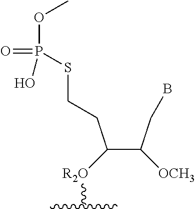

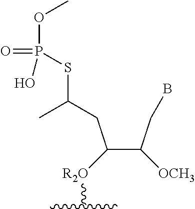

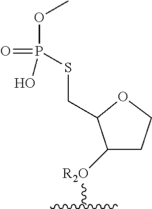

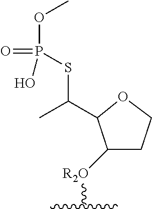

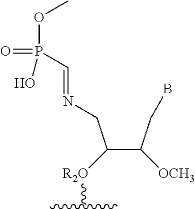

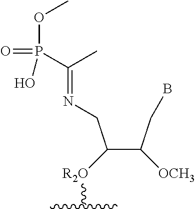

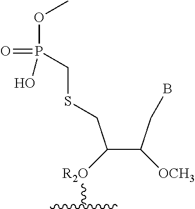

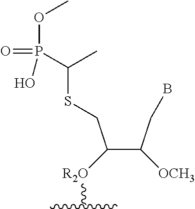

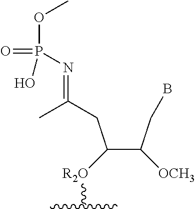

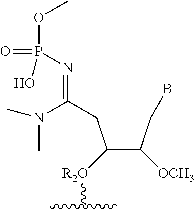

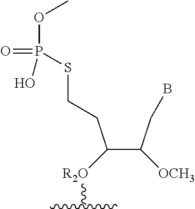

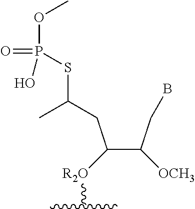

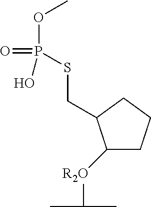

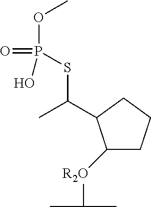

Preferably, the monophosphate derivative has a structure in Table 7.

TABLE 7

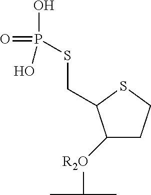

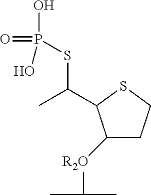

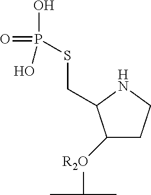

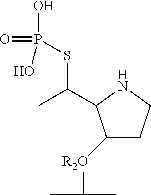

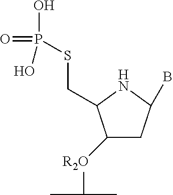

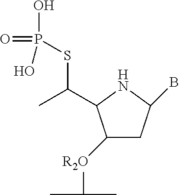

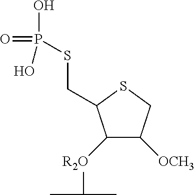

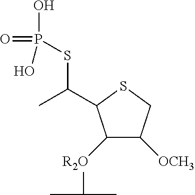

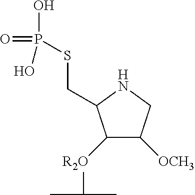

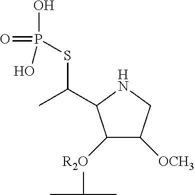

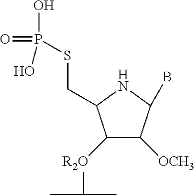

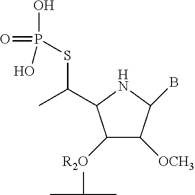

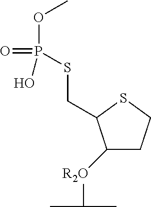

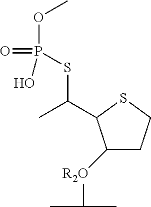

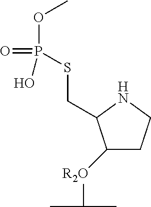

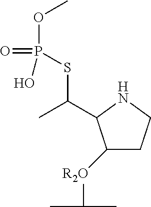

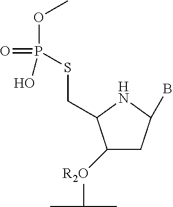

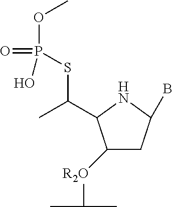

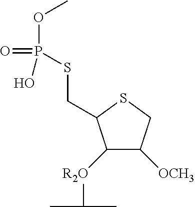

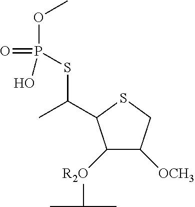

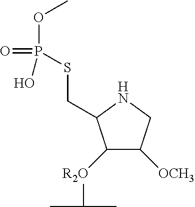

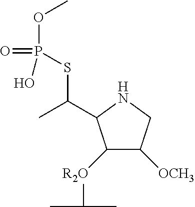

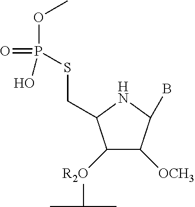

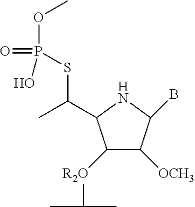

Preferably, nucleotides comprising a monophosphate derivative according to the invention have a structure including, but not limited to, those depicted in Table 8.

TABLE 8

| Nucleotide | |
|---|---|
| monomer # | Structure |
| 1 | 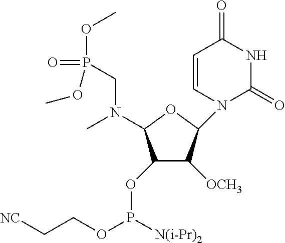 |
| 2 | 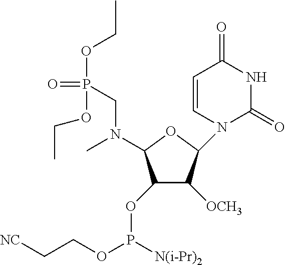 |
| 3 | 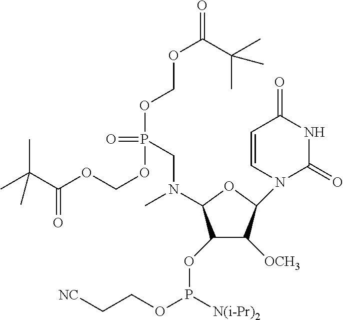 |
| 4 | 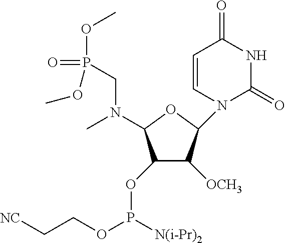 |
| 5 | 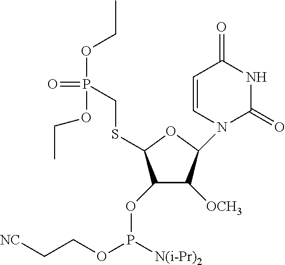 |
| 6 | 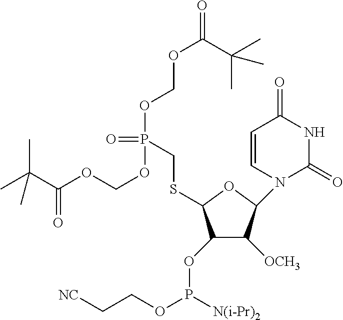 |
| 7 | 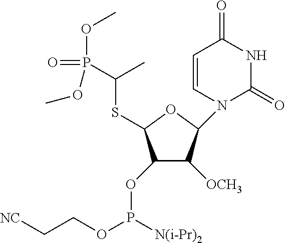 |
| 8 | 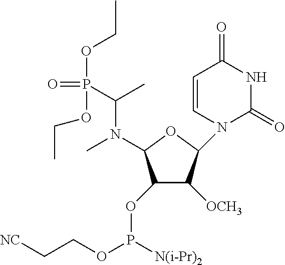 |
| 9 | 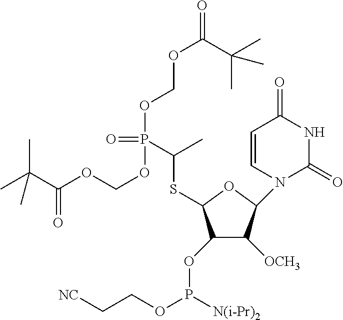 |
| 10 | 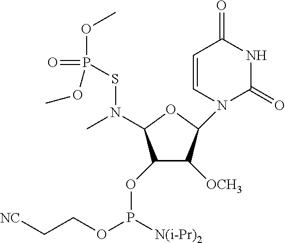 |
| 11 | 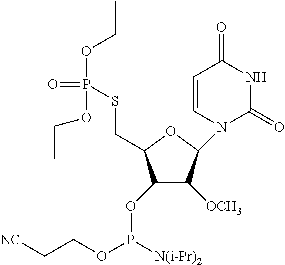 |
| 12 | 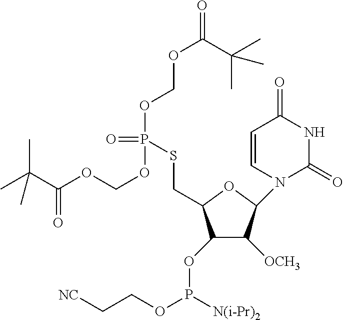 |
| 13 | 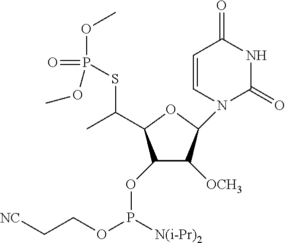 |
| 14 | 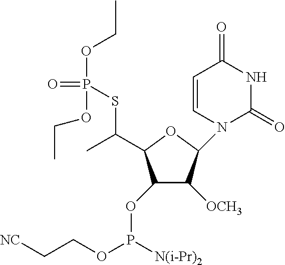 |
| 15 | 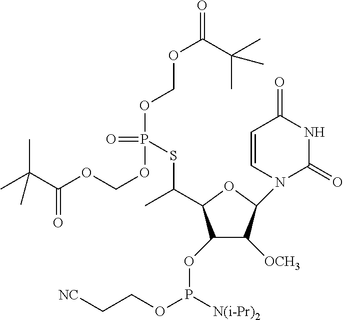 |
| 16 | 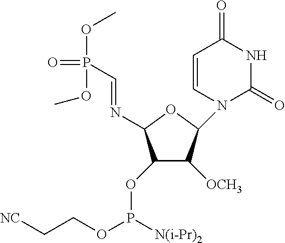 |
| 17 | 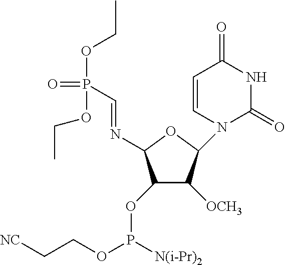 |
| 18 | 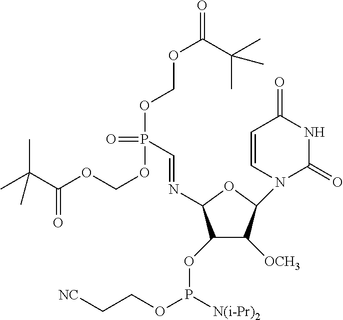 |
| 19 | 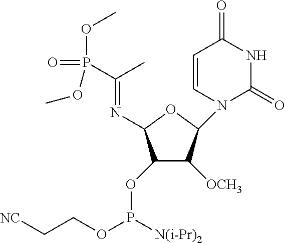 |
| 20 | 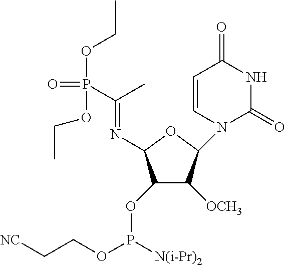 |
| 21 | 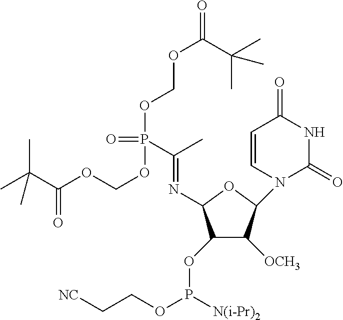 |

Preferably, nucleotides comprising a monophosphate derivatives according to the invention have a structure including, but not limited to, those depicted in Table 9.

TABLE 9

| Nucleotide monomer # | Structure |
|---|---|
| 22 | 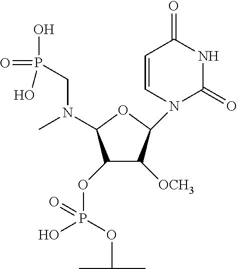 |
| 23 | 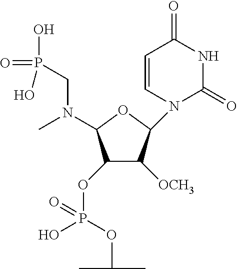 |
| 24 | 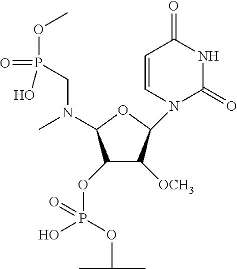 |
| 25 | 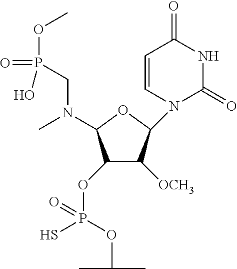 |
| 26 | 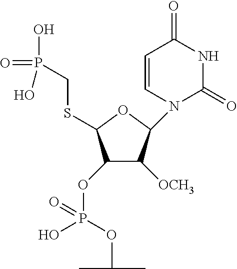 |
| 27 | 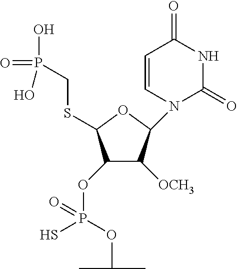 |
| 28 | 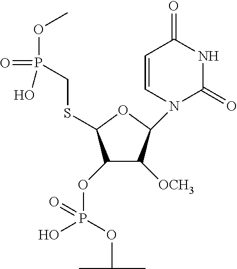 |
| 29 | 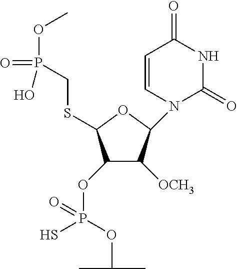 |
| 30 | 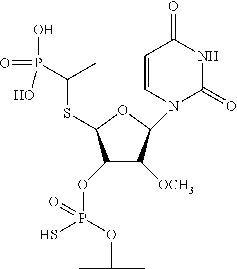 |
| 31 | 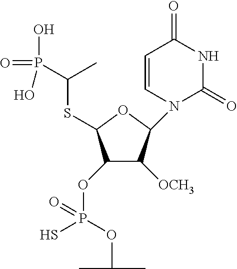 |
| 32 | 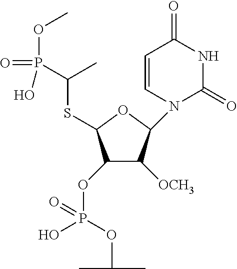 |
| 33 | 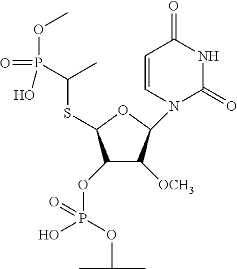 |
| 34 | 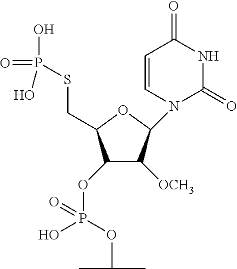 |
| 35 | 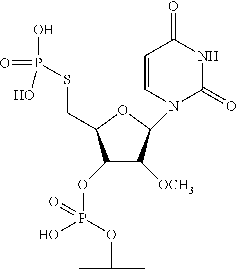 |
| 36 | 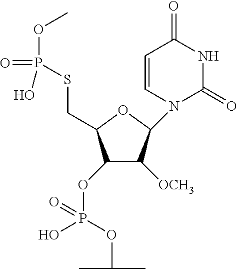 |
| 37 | 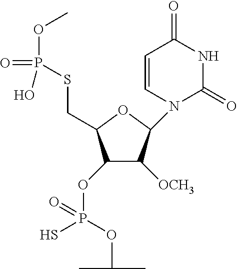 |
| 38 | 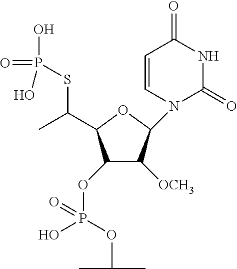 |
| 39 | 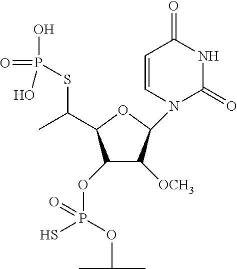 |
| 40 | 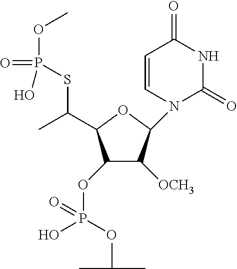 |
| 41 | 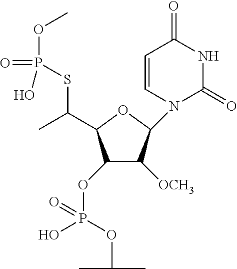 |
| 42 | 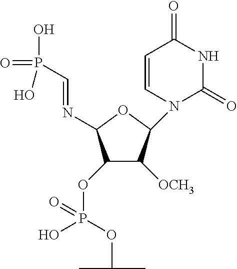 |
| 43 | 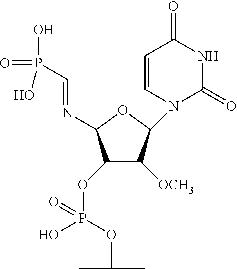 |
| 44 | 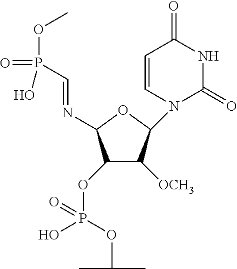 |
| 45 | 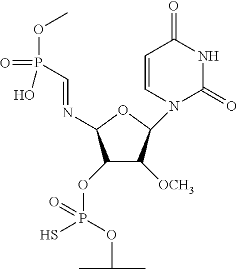 |
| 46 | 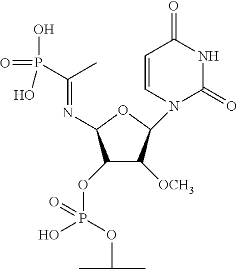 |
| 47 | 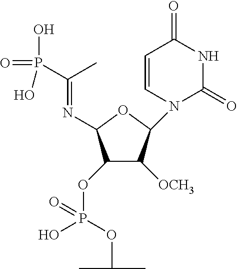 |
| 48 | 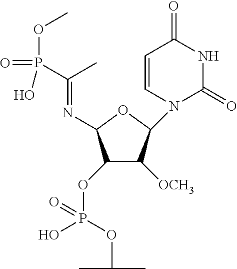 |
| 49 | 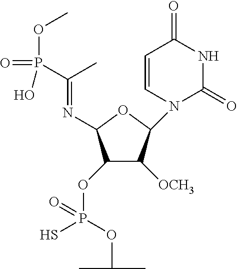 |

In some aspects the invention provides an oligonucleotide comprising a nucleotide or abasic nucleotide comprising a monophosphate derivative as defined herein. Preferably the nucleotide comprising the monophosphate derivative is a 3′-terminal nucleotide or a 5′-terminal nucleotide.

Preferably the nucleotide comprising the monophosphate derivative is a 3′-terminal nucleotide.

Preferably the nucleotide comprising the monophosphate derivative is a 5′-terminal nucleotide.

In embodiments, oligonucleotide comprising a nucleotide comprising a monophosphate derivative of the invention is a gene modulating oligonucleotide (i.e., an oligonucleotide that can modulate the expression of a target gene). Such oligonucleotides can include, but are not limited to, an antisense oligonucleotide, siRNA, a microRNA (miRNA), a miRNA mimic, a piRNA, a hnRNA, a ncRNA, a snRNA, a sgRNA, an esiRNA, an shRNA, or a lncRNA.

Preferably, an oligonucleotide comprising the 3′ or 5′-terminal nucleotide comprising the monophosphate derivative of the invention has a structure represented by formula (C) or (D):

5′-NₘNₓN₁₈N₁₇N₁₆N₁₈N₁₄N₁₃N₁₂N₁₁N₁₀N₉N₈N₇N₆N₅N₄N₃N₂N₁-3′ (formula (C)

5′-NₓN₁₈N₁₇N₁₆N₁₈N₁₄N₁₃N₁₂N₁₁N₁₀N₉N₈N₇N₆N₅N₄N₃N₂N₁Nₘ-3′ (formula (D)
wherein
N₁-N₁₈ represents any modified or unmodified nucleotide;
Nₓ represents any modified or unmodified nucleotide, wherein x=0-6; and
Nₘ represents any nucleotide or abasic nucleotide comprising the monophosphate derivative of the invention.

Preferably, the oligonucleotide comprising the monophosphate derivative of the invention is an antisense oligonucleotide.

Preferably the 5′terminal nucleotide of the antisense oligonucleotide comprises the monophosphate derivative of the invention.

In some aspects, the invention relates to a double stranded nucleic acid comprising: a first oligonucleotide and a second oligonucleotide, wherein at least one of the first and second oligonucleotide comprises a monophosphate derivative according to the invention. In embodiments, the first and second oligonucleotide each independently comprises 10 to 30 linked nucleosides, preferably 19 to 25 nucleotides. In embodiments, at least one of the first and second oligonucleotide comprises 10 to 30 linked nucleosides, preferably 19 to 25 nucleotides.

In embodiments, the first oligonucleotide comprises the monophosphate derivative according to the invention. In embodiments, the second oligonucleotide comprises the monophosphate derivative according to the invention. In embodiments, the first oligonucleotide and the second oligonucleotide comprise the monophosphate derivative of the invention. In embodiments, the monophosphate derivative of the invention is the 3′ terminal nucleotide or the 5′ terminal nucleotide. Preferably, the monophosphate derivative of the invention is the 3′ terminal nucleotide. Preferably, the monophosphate derivative of the invention is the 5′ terminal nucleotide In some aspects, the invention relates to a double-stranded RNA molecule (siRNA), or a pharmaceutically acceptable salt thereof, comprising a sense strand and an antisense strand, wherein at least one of the sense strand and the antisense strand comprises 10 to 30 nucleotides, preferably 19 to 25 nucleotides. The antisense strand comprises a sequence that is complementary to a target mRNA and has the ability to induce degradation of the target mRNA.

The sense strand has a sequence that is complementary to the antisense strand.

Preferably, the siRNA comprises the monophosphate derivative as described herein as the 3′ terminal nucleotide or the 5′-terminal nucleotide to the antisense strand or the sense strand.

Preferably, the monophosphate derivative as described herein is the 3′ terminal nucleotide of the sense strand. Preferably, the monophosphate derivative as described herein is the 3′ terminal nucleotide of the antisense strand. Preferably, the monophosphate derivative as described herein is the 5′ terminal nucleotide of the sense strand. Preferably, the monophosphate derivative as described herein is the 5′ terminal nucleotide of the antisense strand.

Any single or double stranded oligonucleotide comprising the monophosphate derivative of the invention can further comprise one or more additional sugar, base, or backbone modifications, or combinations thereof. In embodiments, any single or double stranded oligonucleotide comprising the monophosphate derivative of the invention can further comprise one or more 2′-F modified nucleotides, 2′-OMe modified nucleotides, or combinations thereof.

The linking group (L) links the monophosphate derivative of the invention to any single or double stranded oligonucleotide.

The chemical modification of oligonucleotides to introduce a 5′-terminal monophosphate derivative of the invention can, under specific conditions, stabilize the nucleic acid structure and reduce the risk of degradation in various biological media (such as nucleases and phosphatases).

For example, synthetic oligonucleotides are often terminated with a 5′- or 3′-hydroxyl group. The terminal hydroxyl group can be replaced with a phosphate group, which can be used to link the oligonucleotide directly to another nucleic acid using linkers, adapters. Furthermore, modification of the 5′-terminal phosphate group enhances the interaction between certain nucleic acid inhibitor molecules and Ago2. However, oligonucleotides with 5′-monophosphate groups are often susceptible to degradation by phosphatases or other enzymes, which limits their bioavailability in the body.

Without wishing to be bound to any particular theory, the terminal monophosphate derivative of the invention provide a functional role similar to the phosphate group but will be resistant against phosphatases and other enzymes while minimizing negative effects on oligonucleotide function; such as maintaining or improving RISC binding affinity and intrinsic potency.

Examples of oligonucleotides comprising a monophosphate derivative of the invention include, but are not limited to, the oligonucleotides of Table 10. Oligonucleotides directed to any other target of interest are well within the skill of one in the art.

TABLE 10
Sequences of modified siRNA comprising a 5′-terminal monophosphate derivative

| | |
|---|---|
| DP-01 | 5′-mCsmAsmUmUmUmUAfmAUfCfCfmUmCmAmCmUmCmUmAsmAsmA-3′ |
| | 5′-N1sUfsmUmAmGAfmGUfGfmAmGmGmAUfmUAfmAmAmAmUmGsmAsmG-3′ |
| DP-02 | 5′-mCsmAsmUmUmUmUAfmAUfCfCfmUmCmAmCmUmCmUmAsmAsmA-3′ |
| | 5′-N2sUfsmUmAmGAfmGUfGfmAmGmGmAUfmUAfmAmAmAmUmGsmAsmG-3′ |
| DP-03 | 5′-mCsmAsmUmUmUmUAfmAUfCfCfmUmCmAmCmUmCmUmAsmAsmA-3′ |
| | 5′-N3sUfsmUmAmGAfmGUfGfmAmGmGmAUfmUAfmAmAmAmUmGsmAsmG-3′ |
| DP-04 | 5′-mCsmAsmUmUmUmUAfmAUfCfCfmUmCmAmCmUmCmUmAsmAsmA-3′ |
| | 5′-N4sUfsmUmAmGAfmGUfGfmAmGmGmAUfmUAfmAmAmAmUmGsmAsmG-3′ |
| DP-05 | 5′-mCsmAsmUmUmUmUAfmAUfCfCfmUmCmAmCmUmCmUmAsmAsmA-3′ |
| | 5′-N5sUfsmUmAmGAfmGUfGfmAmGmGmAUfmUAfmAmAmAmUmGsmAsmG-3′ |
| DP-06 | 5′-mCsmAsmUmUmUmUAfmAUfCfCfmUmCmAmCmUmCmUmAsmAsmA-3′-L |
| | 5′-N1sUfsmUmAmGAfmGUfGfmAmGmGmAUfmUAfmAmAmAmUmGsmAsmG-3′ |
| DP-07 | 5′-mCsmAsmUmUmUmUAfmAUfCfCfmUmCmAmCmUmCmUmAsmAsmA-3′-L |
| | 5′-N2sUfsmUmAmGAfmGUfGfmAmGmGmAUfmUAfmAmAmAmUmGsmAsmG-3′ |
| DP-08 | 5′-mCsmAsmUmUmUmUAfmAUfCfCfmUmCmAmCmUmCmUmAsmAsmA-3′-L |
| | 5′-N3sUfsmUmAmGAfmGUfGfmAmGmGmAUfmUAfmAmAmAmUmGsmAsmG-3′ |
| DP-09 | 5′-mCsmAsmUmUmUmUAfmAUfCfCfmUmCmAmCmUmCmUmAsmAsmA-3′-L |
| | 5′-N4sUfsmUmAmGAfmGUfGfmAmGmGmAUfmUAfmAmAmAmUmGsmAsmG-3′ |
| DP-10 | 5′-mCsmAsmUmUmUmUAfmAUfCfCfmUmCmAmCmUmCmUmAsmAsmA-3′-L |
| | 5′-N5sUfsmUmAmGAfmGUfGfmAmGmGmAUfmUAfmAmAmAmUmGsmAsmG-3′ |

In Table 10, the capital letters C, G, U, and A represent the base composition of the nucleotide. The lowercase letter g, t, and a represent the base composition of the 2′-deoxy-nucleotide. The lowercase letter m indicates that the adjacent nucleotide to the right of the letter m is a 2′-methoxy modified nucleotide (i.e., the pentose 2′-OH of the nucleotide is replaced by methoxy). The lowercase letter f indicates that the adjacent nucleotide to the left of the letter f is a 2′-fluorine modified nucleotide (i.e., the pentose 2′-OH of the nucleotide is replaced by fluorine). The lowercase letter s indicates that the two nucleotides adjacent to the letter s are connected by a thiophosphate diester bond (i.e., the non-bridging oxygen atom in the phosphate diester bond is replaced by a sulfur atom). There are no other letters between the two adjacent nucleotides on the left and right, indicating a phosphodiester bond connection. N1 through N5 and L are shown below:

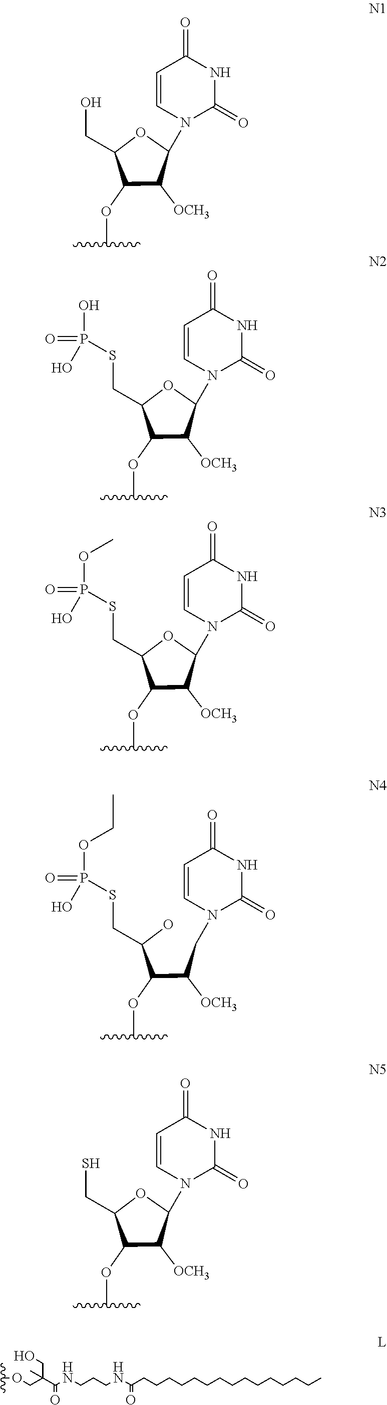

According to some embodiments of the present disclosure, the oligonucleotide comprising the monophosphate derivative of the invention is conjugated to a ligand comprising a structure shown by formula (IX-1) or formula (X-1):

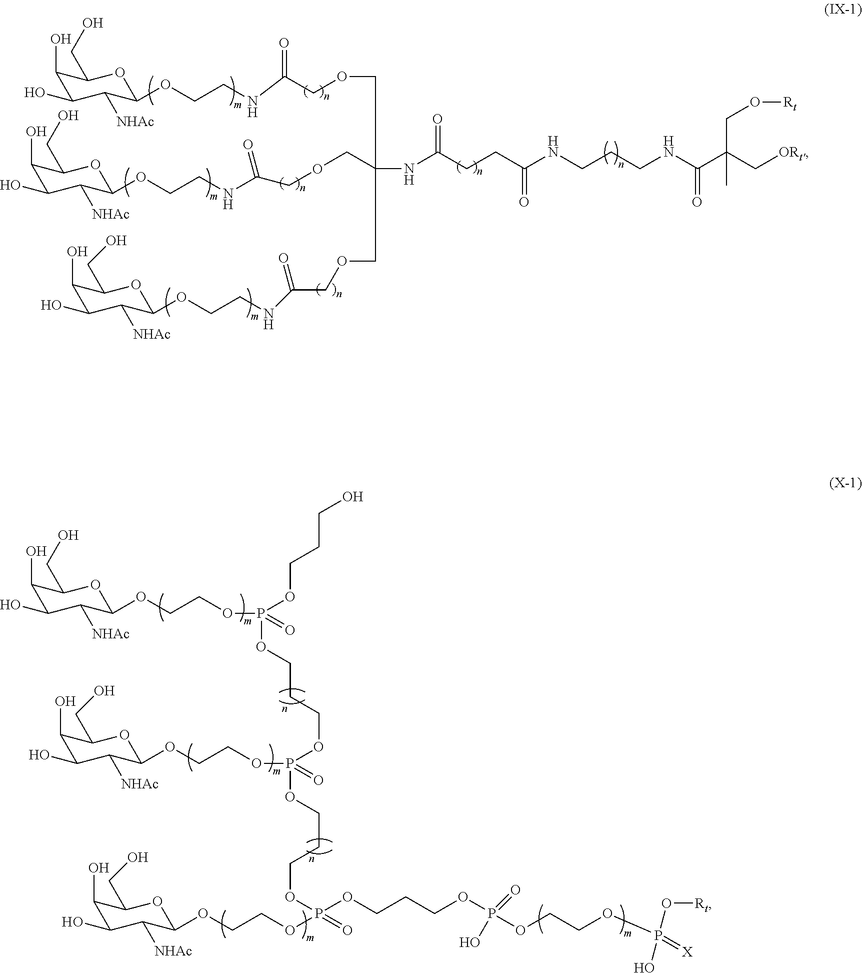

wherein m and n on formula (IX-1) or formula (X-1) are each independently an integer of 0-6; Rₜ and R_{t′} are each independently selected from H, hydroxyl protecting group, oligonucleotide comprising a monophosphate derivative of the invention as described herein (Nu), solid phase carrier, C₂-C₆ carboxyl, carboxylate or amide group; wherein the hydroxyl protecting group is one selected from the group consisting of 4,4′-dimethoxytrityl, 4-methoxytrityl, trityl, t-butyldimethylsilyl, 4-oxopentanoyl, 2-cyanoethyl, 4-pentenoyl and acyloxyalkyl group. Preferably, Rₜ and R_{t′} are an oligonucleotide comprising a monophosphate derivative of the invention (Nu) as described herein.

According to some embodiments of the present disclosure, the conjugate has a structure represented by formula (601), (602), (603), (604), (605), (606) or (607):

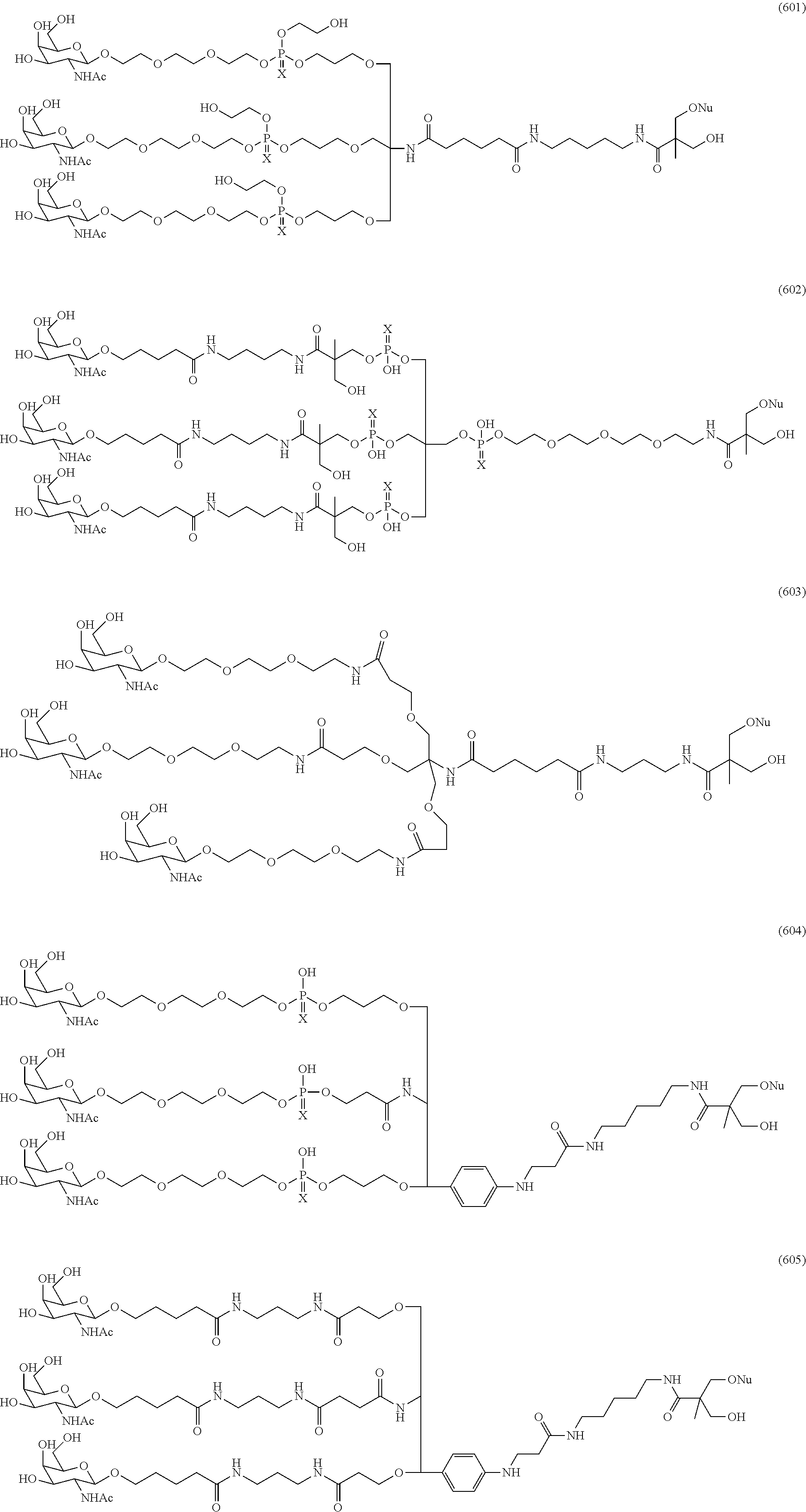

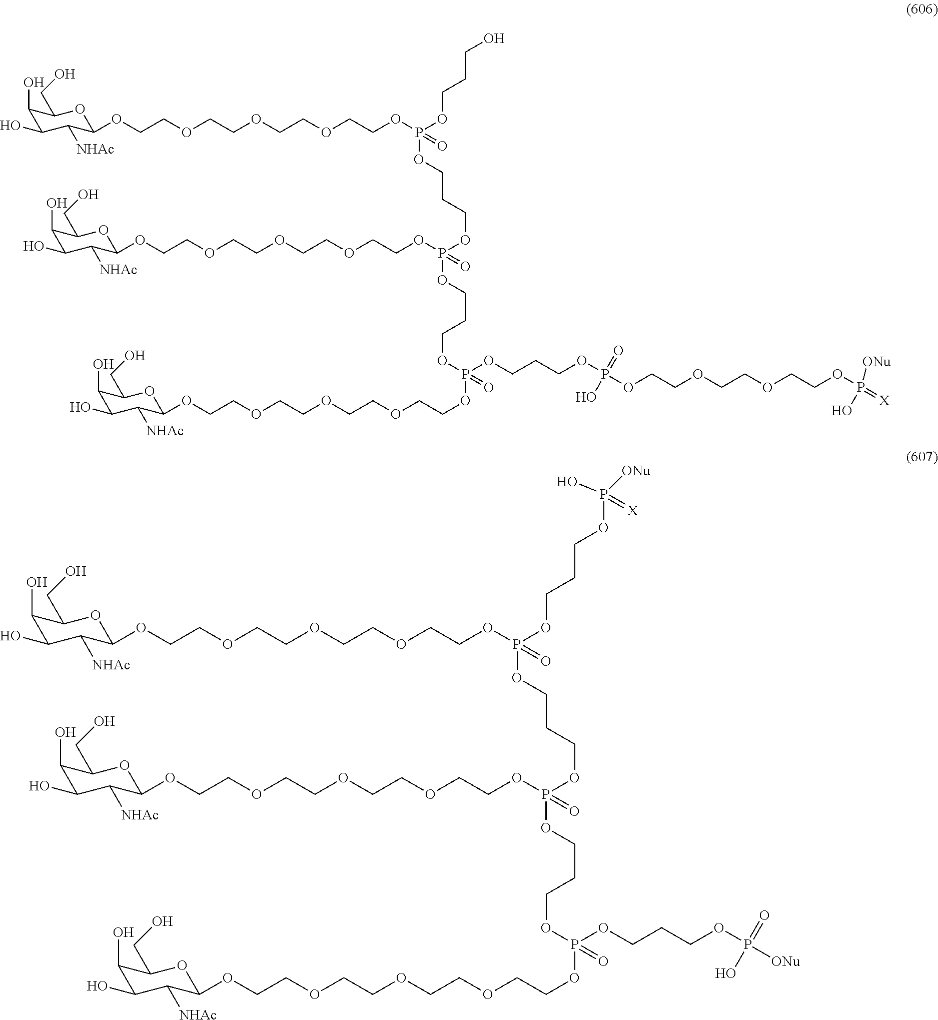

wherein X on formula (601), (602), (603), (604), (605), (606) or (607) is O or S; and Nu is an oligonucleotide comprising the monophosphate derivative of the invention as described herein.

In the present disclosure, a conjugate with the structure shown by formula (603) may be obtained by contacting the compound shown by formula (603A) with an anhydride (e.g., succinic anhydride) to obtain the corresponding carboxylate (603B), the carboxylate (603B) is further connected to a solid phase to obtain a compound (603C), which is further attached on an active group Nu.

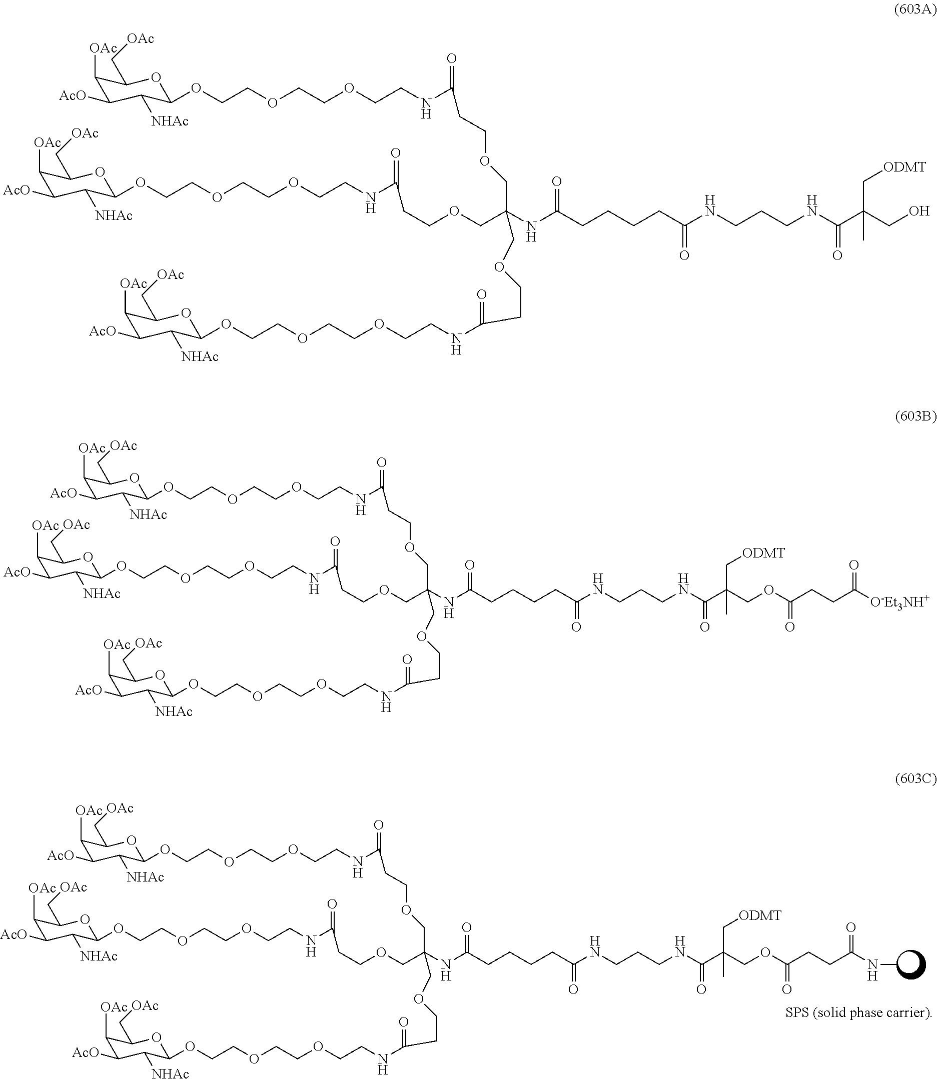

Preferably, the conjugated oligonucleotide comprises the structure:

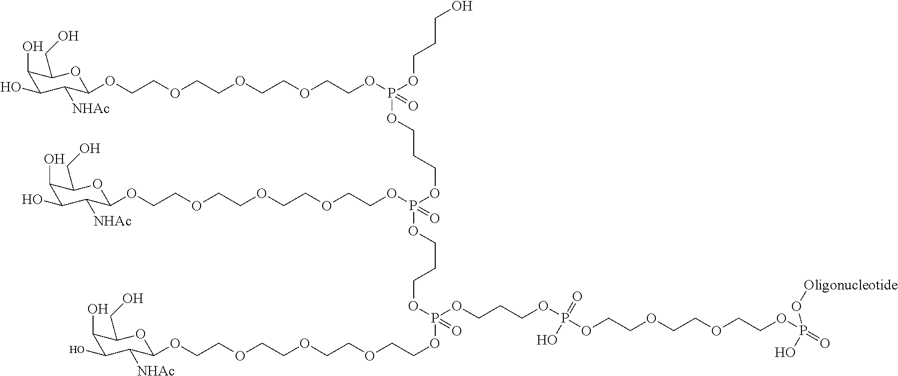

Preferably, the conjugated oligonucleotide comprises the structure:

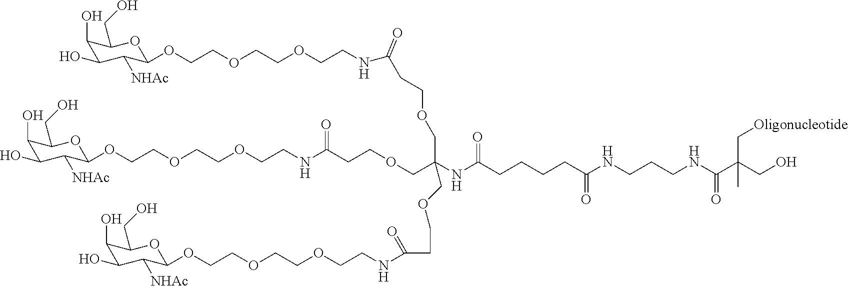

Preferably, the conjugated oligonucleotide comprises the structure:

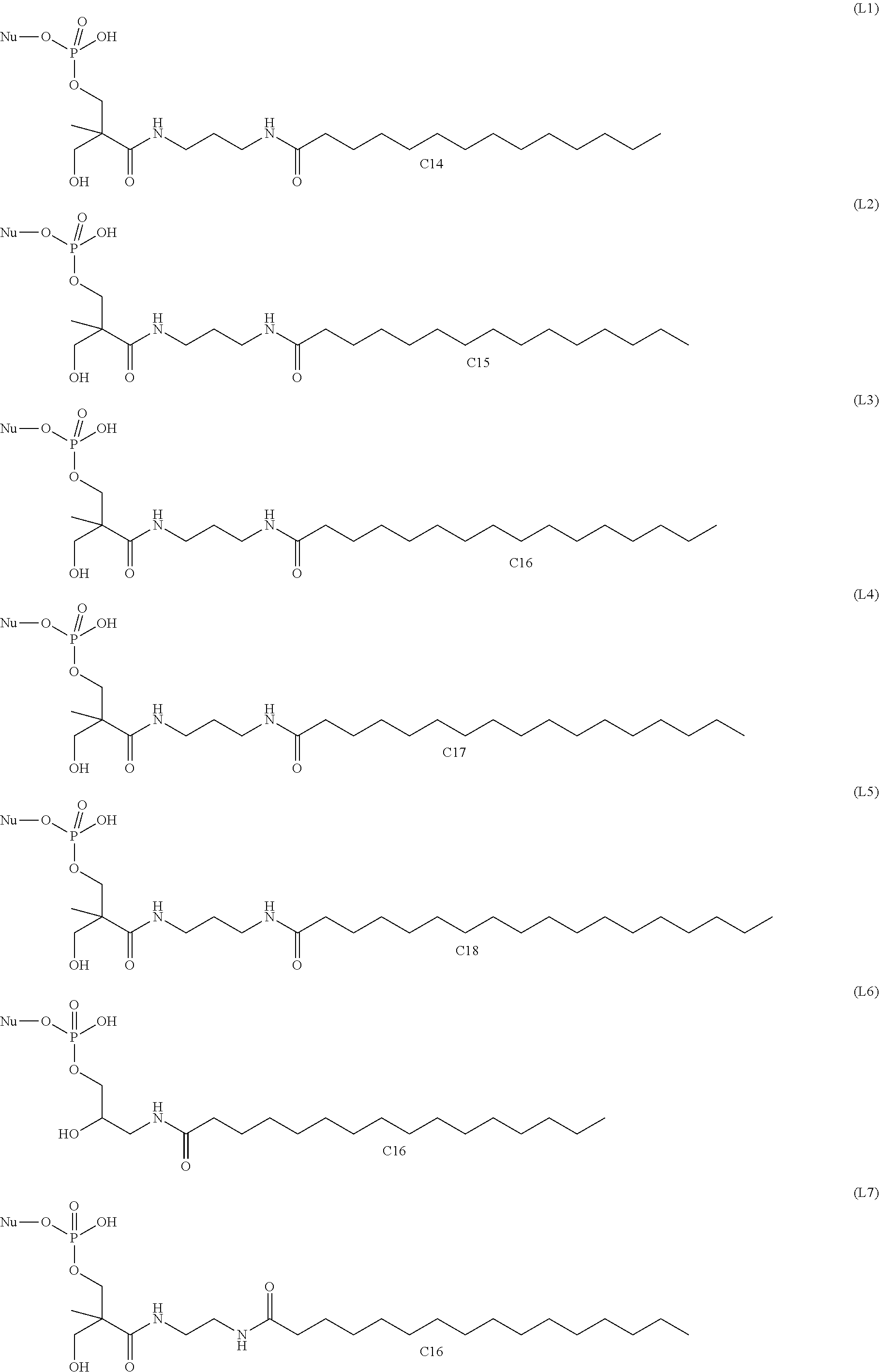

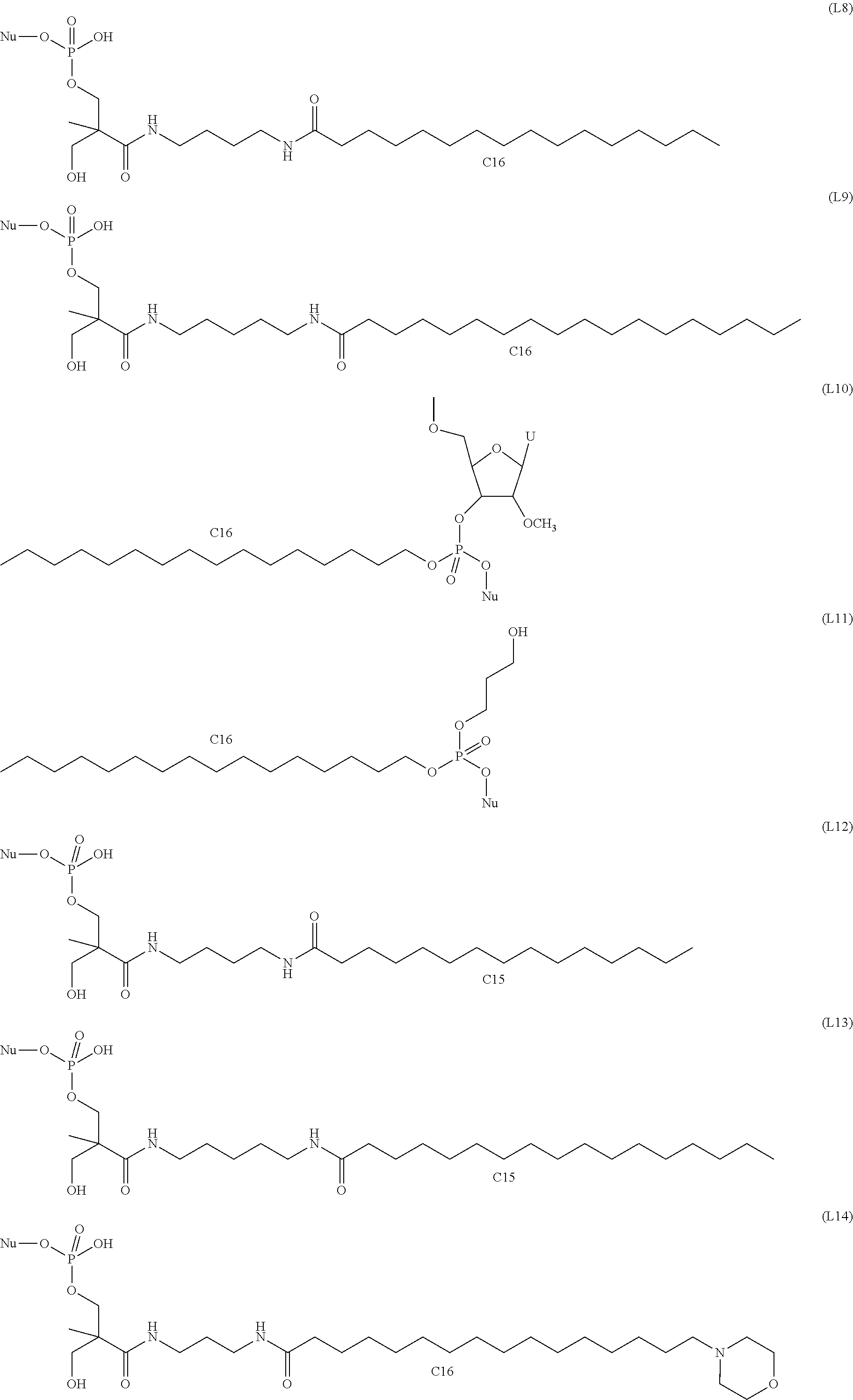

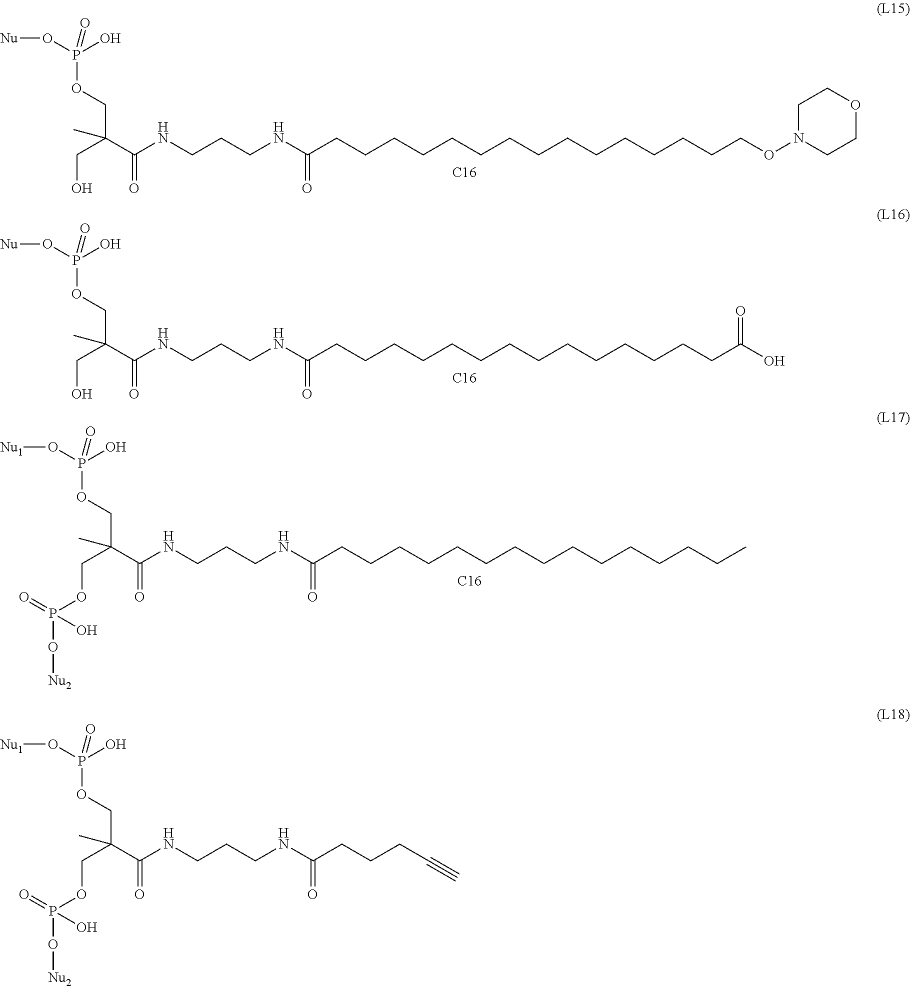

Among them, Nu, Nu1, and Nu2 independently represent a specific and independent oligonucleotide comprising a monophosphate derivative as described herein.

Synthetic route A for preparation of compound (A10):

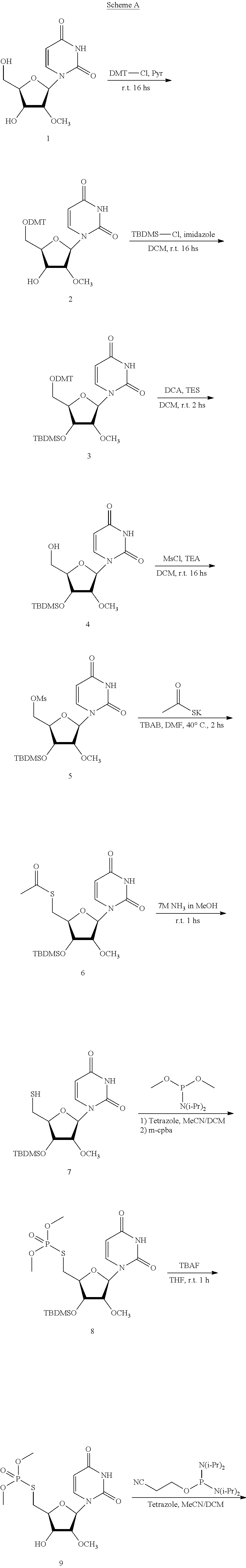

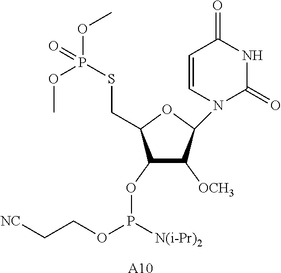

### EXAMPLES

Knockdown of SOD1 with an siRNA comprising a monophosphate derivative according to the invention.

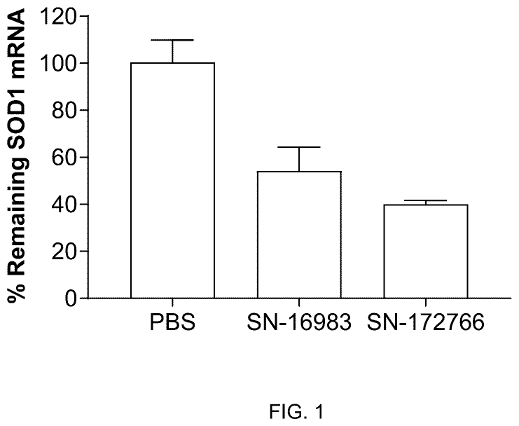

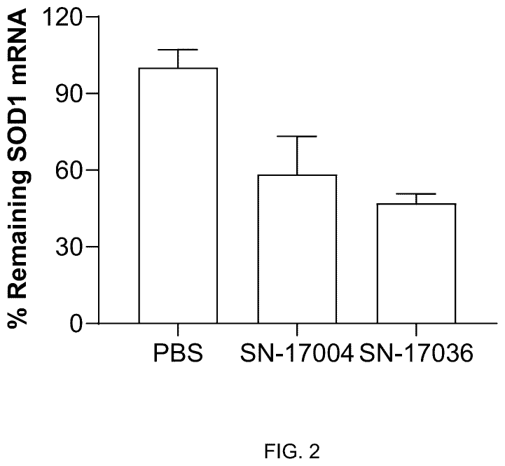

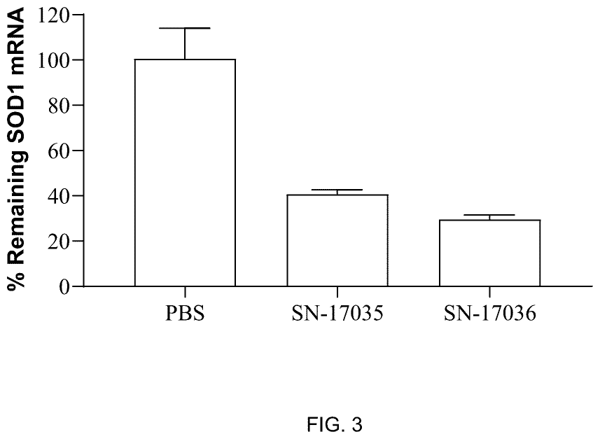

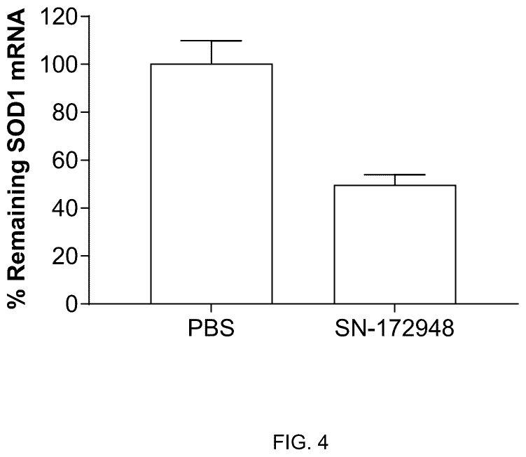

5 groups of rats (n=3) were intrathecally injected with 0.6 mg of an siRNA from Table 11, Table 12, Table 13, or Table 14 or PBS on day 0 and brain sections were collected on day 7. Total RNA was isolated with TRIzol® reagent. cDNA was synthesized by reverse transcription PCR (RT-PCR). Quantitative-PCR (qPCR) was employed to determine the expression of rat SOD1. Briefly, 10 mL of TaqMan Advanced Fast Master mix (Thermo Fisher, Waltham, MA) was mixed with 0.5 mL of 20× rat SOD1 primer probe (Rn00566938_m1, Thermo Fisher), 0.17 mL of 60× rat HPRT1 primer probe (Rn01527840_m1, Thermo Fisher) and 1 mL H2O. 1 mL of cDNA was added into the mixture and subject to qPCR. The expression of rat SOD1 was compared to the SOD1 expression of PBS group using rat HPRT1 as an endogenous control. Results for the siRNAs of Table 11 are shown in FIG. 1. Results for the siRNAs of Table 12 are shown in FIG. 2. Results for the siRNAs of Table 13 are shown in FIG. 3. Results for the siRNAs of Table 14 are shown in FIG. 4.

TABLE 11
siRNAs targeting rat SOD1 gene

| | Sense strand (5′-3′) | Antisense strand (5′-3′) |
|---|---|---|
| SN-16983 | CsAsUUUUAfAUfCfCfUCACUCUAsAsA-L3 | UsUfsUAGAfGUfGfAGGAUfUAfAAAUGsAsG |
| SN-172766 | CsAsUUUUAfAUfCfCfUCACUCUAsAsA-L3 | N3-UsUfsUAGAfGUfGfAGGAUfUAfAAAUGsAsG |

Legend: f = ; s = ; L3 = ; N3 is the monophosphate derivative of formula N3.

TABLE 12

| | Sense strand (5′-3′) | Antisense strand (5′-3′) |
|---|---|---|
| SN-17004 | CsAsUUUUAfAUfCfCfUCACUCUAsAsA-L17- | UsUfsUAGAfGUfGfAGGAUfUAfAAAUGsAsG |
| | GsTsCsGsCsCsCsTsTsCsAsGsCsAsCsG | |
| SN-17036 | CsAsUUUUAfAUfCfCfUCACUCUAsAsA-L17- | N3-UsUfsUAGAfGUfGfAGGAUfUAfAAAUGsAsG |
| | GsTsCsGsCsCsCsTsTsCsAsGsCsAsCsG | |

Legend: f = ; s = ; L3 = ; N3 is the monophosphate derivative of formula N3.

TABLE 13

| | Sense strand (5′-3′) | Antisense strand (5′-3′) |
|---|---|---|
| SN-17035 | CsAsUUUUAfAUfCfCfUCACUCUAsAsA-L19- | UsUfsUAGAfGUfGfAGGAUfUAfAAAUGsAsG |
| | GsTsCsGsCsCsCsTsTsCsAsGsCsAsCsG | |
| SN-172957 | CsAsUUUUAfAUfCfCfUCACUCUAsAsA-L19- | N3-UsUfsUAGAfGUfGfAGGAUfUAfAAAUGsAsG |
| | GsTsCsGsCsCsCsTsTsCsAsGsCsAsCsG | |

Legend: f = ; s = ; L3 = ; N3 is the monophosphate derivative of formula N3.

TABLE 14

| | Sense strand (5′-3′) | Antisense strand (5′-3′) |
|---|---|---|
| SN-172948 | CsAsUUUUAfAUfCfCfUCACUCUAsAsA-L3 | N4-UsUfsUAGAfGUfGfAGGAUfUAfAAAUGsAsG |

Legend: f = ; s = ; L3 = ; N4 is the monophosphate derivative of formula N4.

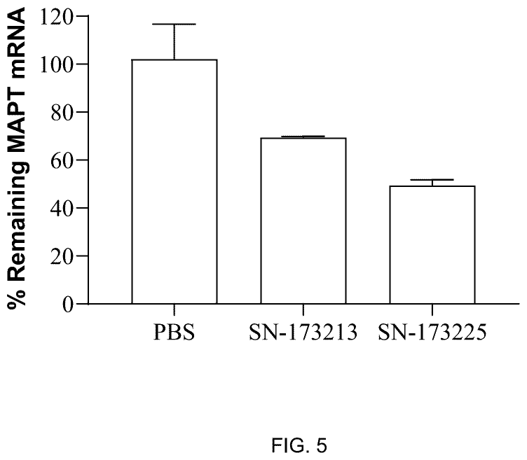

To determine if an siRNA comprising a monophosphate derivative of the invention is applicable to other targets, 150 mg of an siRNA targeting human MAPT (from Table 15) or PBS were intracranially injected into human MAPT transgenic mice (n=3) on day 0. Brain samples were collected on day 30 and RNA was processed as previously described. qPCR was employed to determine the expression of MAPT (human MAPT primer-probe: Hs00902194_m1; mouse HPRT primer-probe: Mm03024075_m1). The results are shown in FIG. 5.

TABLE 15

| siRNA No. | Sense strand (5′-3′) | Antisense strand (5′-3′) |
|---|---|---|
| SN-173213 | GsAsGGAAAfUAfAfAfAAGAUUGASAsA-L19- | UsUfsUCfAAfUCUUUUUAfUUfUCCUCsCsG |
| | CsGsTsTsTsTsCsTsTsAsCsCsAsCsCsC | |
| SN-173225 | GsAsGGAAAfUAfAfAfAAGAUUGAsAsA-L19- | N3-UsUfsUCfAAfUCUUUUUAfUUfUCCUCsCsG |
| | CsGsTsTsTsTsCsTsTsAsCsCsAsCsCsC | |

Legend: f = ; s = ; L3 = ; N3 is the monophosphate derivative of formula N3.

LC-MS Analysis of SiRNAs shown in Tables 11-15

| | |
|---|---|
| SN-16983 | ss: 5′-CsAsUUUUAfAUfCfCfUCACUCUAsAsA-L3 |
| | Cacld MS: 7324.21; Found MS: 7326.1 |
| | as: 5′-UsUfsUAGAfGUfGfAGGAUfUAfAAAUGsAsG |
| | Cacld MS: 7775.2; Found MS: 7773.0 |
| SN-172766 | ss: 5′-CsAsUUUUAfAUfCfCfUCACUCUAsAsA-L3 |
| | Cacld MS: 7324.21; Found MS: 7326.1 |
| | as: N3-5′-UsUfsUAGAfGUfGfAGGAUfUAfAAAUGsAsG |
| | Cacld MS: 7885.24; Found MS: 7885.6 |
| SN-17004 | ss: 5′-CsAsUUUUAfAUfCfCfUCACUCUAsAsA-L17-GsTsCsGsCsCsCsTsTsCsAsGsCsAsCsG |
| | Cacld MS: 13136.0; Found MS: 13135.8: |
| | as: 5′-UsUfsUAGAfGUfGfAGGAUfUAfAAAUGsAsG |
| | Cacld MS: 7775.2; Found MS: 7773.0 |
| SN-17036 | ss: 5′-CsAsUUUUAfAUfCfCfUCACUCUAsAsA-L17-GsTsCsGsCsCsCsTsTsCsAsGsCsAsCsG |
| | Cacld MS: 13136.0; Found MS: 13135.8 |
| | as: N3-5′-UsUfsUAGAfGUfGfAGGAUfUAfAAAUGsAsG |
| | Cacld MS: 7885.24; Found MS: 7885.6 |
| SN-17035 | ss: 5′-CsAsUUUUAfAUfCfCfUCACUCUAsAsA-L19-GsTsCsGsCsCsCsTsTsCsAsGsCsAsCsG |
| | Cacld MS: 13170.0; Found MS: 13164.9 |
| | as: 5′-UsUfsUAGAfGUfGfAGGAUfUAfAAAUGsAsG |
| | Cacld MS: 7775.2; Found MS: 7773.0 |
| SN-172957 | ss: 5′-CsAsUUUUAfAUfCfCfUCACUCUAsAsA-L19-GsTsCsGsCsCsCsTsTsCsAsGsCsAsCsG |
| | Cacld MS: 13170.0 Found MS: 13164.9 |
| | as: N3-5′-UsUfsUAGAfGUfGfAGGAUfUAfAAAUGsAsG |
| | Cacld MS: 7885.24; Found MS: 7885.6 |
| SN-173213 | ss: 5′-GsAsGGAAAfUAfAfAfAAGAUUGAsAsA-L19-CsGsTsTsTsTsCsTsTsAsCsCsAsCsCsC |
| | Cacld MS: 13434.4; Found MS: 13427.8 |
| | as: 5′-UsUfsUCfAAfUCUUUUUAfUUfUCCUCsCsG |
| | Cacld MS: 7408.8; Found MS: 7411.5 |
| SN-173225 | ss: 5′-GsAsGGAAAfUAfAfAfAAGAUUGAsAsA-L19-CsGsTsTsTsTsCsTsTsAsCsCsAsCsCsC |
| | Cacld MS: 13434.4; Found MS: 13427.8 |
| | as: N3-5′-UsUfsUCfAAfUCUUUUUAfUUfUCCUCsCsG |
| | Cacld MS: 7519.9; Found MS: 7517.6 |
| SN-172948 | ss: 5′-GsAsACCCGfAAfAGfCUACUGAACU-TriGalNac |
| | Cacld MS: 8802.5; Found MS: 8801.9 |
| | as: N4-5′-UsGfsUUfCAfGUAGCUUUfCGfGGUUCsUsU |
| | Cacld MS: 7708.07; Found MS: 7708.2 |

### Synthetic Route B for Preparation of Compound (A11, A12):

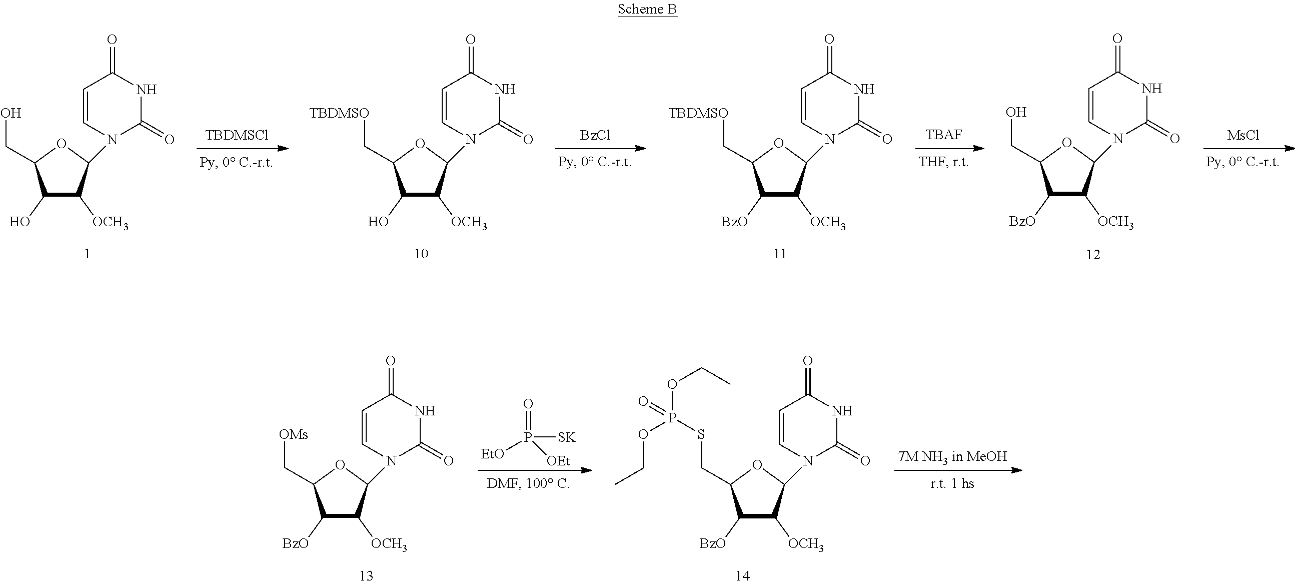

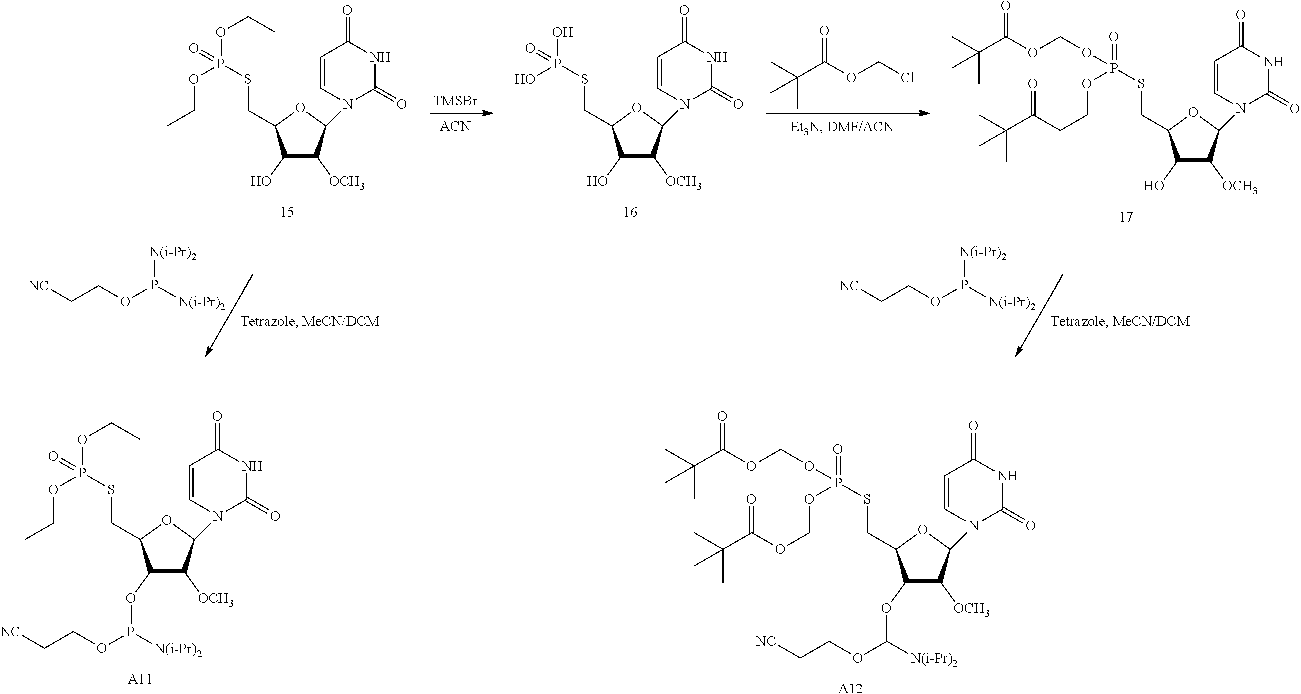

### Synthesis of N3-N4-N5-Oligonucleotide

### Synthesis of 5′-N3 Oligonucleotide by Using A10 Amidite

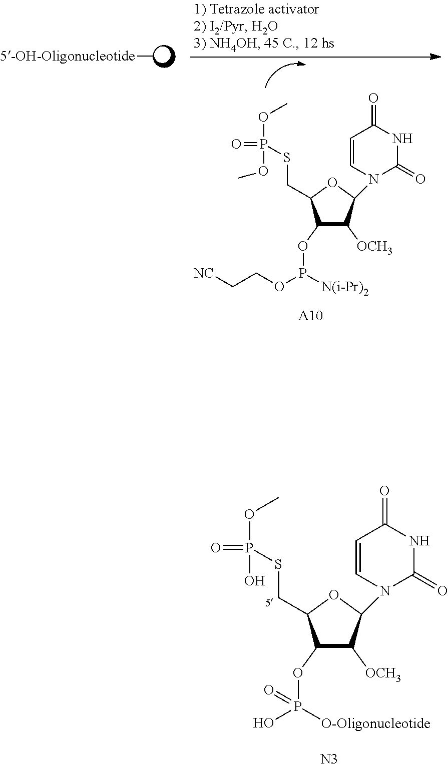

### Synthesis of 5′-N4 Oligonucleotide by Using all Amidite

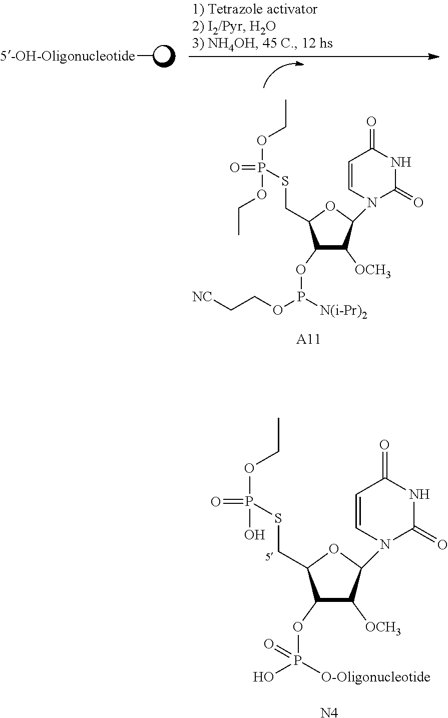

### Synthesis of 5′-N2 or N5 Oligonucleotide by Using A12 Amidite

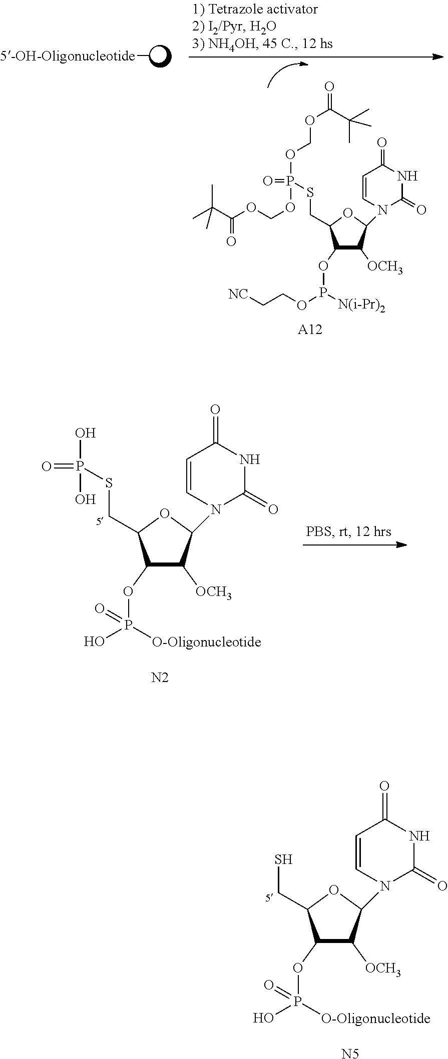

While this invention has been particularly shown and described with references to preferred embodiments thereof, it will be understood by those skilled in the art that various changes in form and details may be made therein without departing from the scope of the invention encompassed by the appended claims.

1. A monophosphate derivative comprising a structure represented by formula (A) or (B): 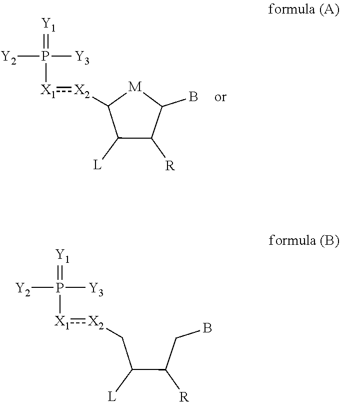
wherein:
X₁ is —CH2-, —CHR^{a}—, —CR^{a}R^{b}—, —NH—, —NR^{c}—, —N═CH—, —N═CR^{d}—, or —S—;
X₂ is —CH2-, —CHR^{a}′—, —CR^{a}′R^{b}′—, —NH—, —NR^{o}′—, —CH═N—, —CR^{d}′═N—, or —S—;
R^{a}, R^{b}, R^{c}, R^{d}, R^{a}′, R^{b}′, R^{c}′, and R^{d}′ are independently selected from C₁-C₁₀ alkyl;
Y₁ is O or S;
Y₂ and Y₃ are independently selected from —OH, —O(CH₂)ᵥOC(O)R^{g}; —SR^{c}, —NHR^{e}, C₁-C₁₀ alkyl, or C₁-C₁₀ alkoxy, —ONa⁺, —SNa⁺, wherein v is 1-6;
B is selected from H, C, O, N or a nucleotide base;
M is selected from O, S, —CH₂, or —NH;
R is selected from H, —OH, —NH₂, halogen, C₁-C₁₀ alkyl, C₁-C₁₀ alkoxy, C₂-C₁₀ alkenyl, —CN, or haloalkyl, —CO₂H, —CO₂R^{e}, or —CONHR^{f};
L is a linking group; and
R^{e}, R^{f}, and R^{g} are independently selected from H, C₁-C₁₀ alkyl, C₁-C₁₀ alkoxy, or C₂-C₁₀ alkenyl.

2. The monophosphate derivative according to claim 1, wherein the monophosphate derivative comprises a structure represented by formula (A1), (A2), (A3) or (A4): 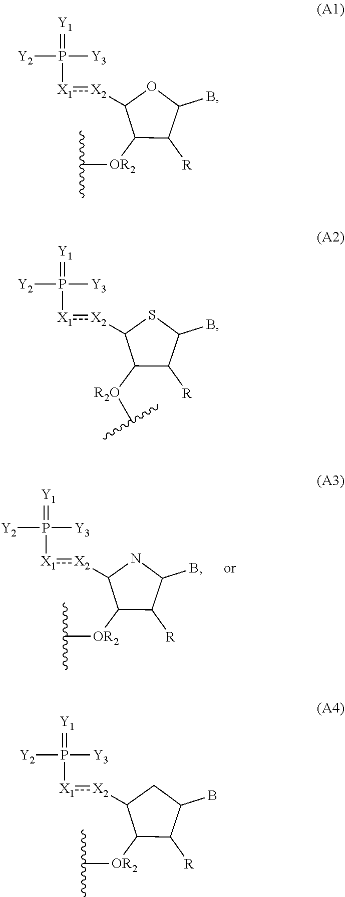
wherein R₂ is a phosphate or an analog represented by formula —P(Q)(W)Z—, wherein Q is O or S, W is hydroxy, C₁-C₆ alkyl, C₁-C₆ alkoxy, SH, NRₕRᵢ, and Z is CHRᵢ, O, or S, wherein each Rh and Rᵢ are independently H, C₁-C₆ alkyl.

3. The monophosphate derivative according to claim 1, wherein the monophosphate derivative comprises a structure represented by formula (B1): 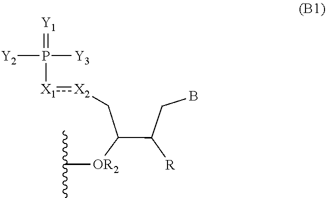
wherein R₂ is a phosphate or an analog represented by formula —P(Q)(W)Z—, wherein Q is O or S, W is hydroxy, C₁-C₆ alkyl, C₁-C₆ alkoxy, SH, NRₕRᵢ, and Z is CHRᵢ, O, or S, wherein each Rh and Rᵢ are independently H, C₁-C₆ alkyl.

4. The monophosphate derivative according to claim 1, wherein L is selected from —OP(═O)(OH)O—, —OP(═O)(SH)O—, —OP(═S)(OH)O—, —OP(═O)(CH₃)O—, —OP(═O)(CH₂CH₃)O—, —OP(═O)(OCH₃)O—, —OP(═O)(OCH₂CH₃)O—, —OP(═O)(NH₂)O—, —OP(═O)(NHCH₃)O—, —OP(═O)(NHCH₂CH₃)O—, —OP(═S)(CH₃)O—, —OP(═S)(CH₂CH₃)O—, —OP(═S)(OCH₃)O—, —OP(═S)(OCH₂CH₃)O—, —OP(═S)(NH₂)O—, —OP(═S)(NHCH₃)O—, —OP(═S)(NHCH₂CH₃)O—, or —OP(═S)(SH)O—.

5. The monophosphate derivative according to claim 1, wherein X₁ is —S—.

6. The monophosphate derivative according to claim 1, wherein Y₁ is O.

7. The monophosphate derivative according to claim 1, wherein Y₂ and Y₃ are independently selected from —OH or C₁-C₁₀ alkoxy.

8. The monophosphate derivative according to claim 1, wherein R is C₁-C₁₀ alkoxy.

9. The monophosphate derivative according to claim 1, wherein the monophosphate derivative is selected from the monophosphate derivative in Table 1 through Table 9.

10. The monophosphate derivative of claim 1, wherein the monophosphate derivative comprises a structure represented by formula N2, N3, or N4: 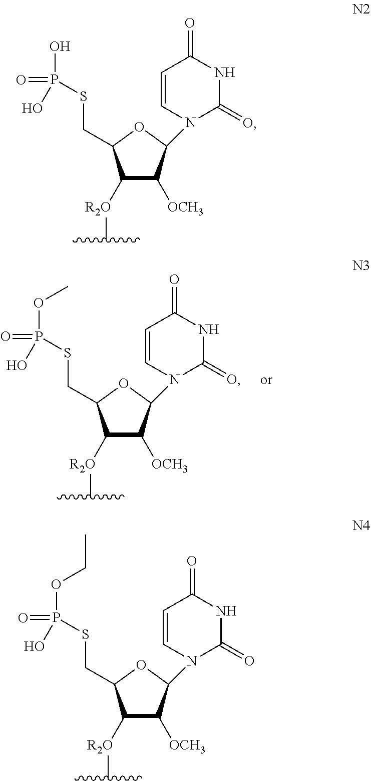
wherein R₂ is a phosphate or an analog represented by formula —P(Q)(W)Z—, wherein Q is O or S, W is hydroxy, C₁-C₆ alkyl, C₁-C₆ alkoxy, SH, NRₕRᵢ, and Z is CHRᵢ, O, or S, wherein each Rh and Rᵢ are independently H, C₁-C₆ alkyl.

11. An oligonucleotide comprising a monophosphate derivative according to claim 1.

12. The oligonucleotide according to claim 11, wherein the linking group links the monophosphate derivative to the oligonucleotide.

13. The oligonucleotide according to claim 11, wherein the oligonucleotide is double stranded comprising a first strand and a second strand, wherein the first strand is a sense strand and the second strand is an antisense strand.

14. The oligonucleotide according to claim 13, wherein the oligonucleotide is double stranded siRNA.

15. The oligonucleotide according to claim 11, wherein the oligonucleotide is single stranded.

16. The oligonucleotide according to claim 11, wherein the monophosphate derivative is at the 5′ terminal end of the oligonucleotide.

17. A compound comprising a ligand conjugated to the oligonucleotide according to claim 11.

18. A double stranded nucleic acid comprising: a first oligonucleotide and a second oligonucleotide, wherein at least one of the first and second oligonucleotide comprises a monophosphate derivative according to claim 1.

19. The double stranded nucleic acid according to claim 18, wherein at least one of the first and second oligonucleotide consists of 10 to 30 linked nucleosides.

20. The double stranded nucleic acid according to claim 18, wherein each of the first and second oligonucleotide independently comprises one or more 2′-F and 2′-OMe modified nucleotides, or combinations thereof.

21. The double stranded nucleic acid according to claim 18, wherein the first oligonucleotide comprises the monophosphate derivative.

22. The double stranded nucleic acid according to claim 1, wherein the second oligonucleotide comprises the monophosphate derivative.

23. The double stranded nucleic acid according to claim 1, wherein the first oligonucleotide and the second oligonucleotide comprises the monophosphate derivative.

24. The oligonucleotide according to claim 18, wherein the linking group links the monophosphate derivative to the first and/or second oligonucleotide.

25. The oligonucleotide according to claim 18, wherein the monophosphate derivative is at the 5′ terminal end of the first and/or second oligonucleotide.
